(19) United States
(12) Patent Application Publication
(10) Pub. No.: US 2004/0202622 A1
(43) Pub. Date: Oct. 14, 2004

(54) EPOSS containing cosmetics and personal care products

(75) Inventor: Murat Quadir, Scotch Plains, NJ (US)

Correspondence Address: LERNER, DAVID, LITTENBERG,, KRUMHOLZ & MENTLIK, 600 SOUTH AVENUE WEST, WESTFIELD, NJ, 07090, US

(73) Assignee: L'OREAL S.A., Paris (FR)

(21) Appl. No.: 10/799,280

(22) Filed: Mar. 12, 2004

Related U.S. Application Data
(60) Provisional application No. 60/455,046, filed on Mar. 14, 2003.

(51) Int. Cl. A61K 7/42; A61K 7/06; A61K 7/11; A61K 7/15

(52) U.S. Cl. 424/59; 424/70.11; 424/73

(57) ABSTRACT

Personal care products including cosmetics, grooming products, products useful for skin and hair treatment, and the like are described including extended polyhedral oligomeric silsesquioxanes having greater than nine silicon atoms in their three-dimensional cage-like structure. Methods of using and methods of making personal care products including these materials are also described.

### CROSS REFERENCE TO RELATED APPLICATIONS

The present application claims the benefit of U.S. Provisional Application No. 60/455,046, filed Mar. 14, 2003, the disclosure of which is incorporated by reference herein.

### BACKGROUND OF THE INVENTION

Designing personal care products such as shampoos, conditioners, make up, nail polish, etc. is a diverse and challenging endeavor. First, one must consider the variety of keratinous materials involved. Hair including facial hair such as beards or mustaches, eyelashes, eyebrows, hair on the scalp, skin on the face, eyelids, lips, arms, etc. and even nails, while all keratinous materials, have very different properties. Second, there is the objective. Is the product a coloring agent or something that provides UV protection? Is the product something that is to be left in the hair or rinsed off of the skin? Should the product be easily removed with water or must a solvent such as nail polish remover be used? All of these issues must be considered in formulating each individual personal care product. But this is by no means an exclusive list of issues.

Within each product type, there are other issues as well. For example, one of the problems facing conditioners is that of “substantivity.” Substantivity is understood in the industry to describe the ability of a conditioning agent, such as a compound containing a quaternary nitrogen or “quat,” to stay in contact with skin or hair for a desired length of time. Identifying a suitable conditioning agent with sufficient conditioning and substantive properties is only a small part of the problem. After such a compound has been discovered, it must be formulated with other ingredients that provide proper viscosity, rinseability, and other desirable properties such as appearance and feel. With each ingredient added, however, the formulation becomes more complex and interactions and incompatibilities are a persistent problem. It is no wonder that the personal care industry is the subject of such intensive research.

The discovery of new and useful compounds that are compatible with a broad range of other ingredients used in the production of personal care products, as well as compositions that can be used in numerous different personal care situations is therefore highly desirable. U.S. patent application Publication No. US2002/0114773A1, which published on Aug. 22, 2002, to Kanji et al. discloses in one embodiment transfer resistant cosmetic compositions, which may also be pliable and comfortable to wear upon application to a substrate. The compositions comprise, more particularly, at least one structuring polymer and at least one silicone resin. The film forming silicone resins described as useful include polymethyl silsesquioxanes formed primarily of polymerized repeating units of CH₃SiO_{3/2}. These polymers are stated as being believed to be in a “cage” (i.e. Formula I) and “ladder” configurations, with the majority of the polymer being in a ladder configuration. See id. at ¶ [0067]. A methyl group may be substituted with a group that is “other than methyl.” Id. at ¶¶ [0056] and [0061]. See also U.S. patent application Pub. No. US2002/0031488A1, EP 0,624,594 and U.S. Pat. Nos. 2,465,188, 5,047,492, 5,246,694 and 5,439,673. Polymethyl silsesquioxanes may also be used in dental bonding agents introduced by Pentron Clinical Technologies.

Polyhedral oligomeric silsesquioxanes are known to exist having more than eight Si atoms in a generally rigid three-dimensional structure. These include those identified in, for example, U.S. Pat. Nos. 6,127,557; 5,858,544; and 2,465,188. Such molecules are also sold by Hybrid Plastics. See Vinyl-POSS cage mixture cat no. OL 1170, EpoxyCyclohexyl-POSS cage mixture, 60%, cat no. EP 0408, and DodecaPhenyl-POSS, cat no. MS 0802.

Silicone resins that are believed to include a mixture of ladder and cage molecules entirely substituted with methyl groups may have been sold by Wacker Chemical Corp., 3306 Sutton Rd., Adrian, Mich. 49221, under the trademark BELSIL® PMS MK, resin MK or Siliconharz MK. It is also believed that these mixtures include a majority of molecules in the ladder configuration. Resins substituted with propyl and phenyl groups are allegedly also available from Wacker under the trademark BELSIL® silicones SPR 45. This molecule is a T resin, has a random structure, and is not believed to be in either a ladder or cage configuration. Lipstick and a hand protection lotion including BELSIL® silicones SPR 45 have also been disclosed.

### SUMMARY OF THE INVENTION

The present invention advances the science of the personal care industry by providing new molecules, new personal care products useful for the treatment of or application to keratinous materials (e.g., hair, facial hair, eyebrows, eyelashes, body hair, skin, lips and nails), as well as methods of applying and/or treating keratinous materials.

In one particularly preferred aspect of the present invention, EPOSS molecules are provided, which have been functionalized, derivatized and/or substituted with at least one group, often a plurality of groups, which render the EPOSS molecule desirable for incorporation in personal care products. “EPOSS,” in accordance with the present invention, means one or a mixture of Extended Polyhedral Oligomeric SilSesquioxane(s). However, not all Extended Polyhedral Oligomeric SilSesquioxanes qualify as EPOSS according to the invention. More specifically, EPOSS molecules in accordance with the present invention contain nine or more Si atoms within their core structures (e.g., not including R groups). They can have, for example purposes only, the general structures of Formulas I and II. The EPOSS molecules are collectively referred to as having a “cage” structure. However, only certain structures, such as, by way of non-limiting examples, those illustrated in Formula Ia and Ic are referred to herein as being “complete cages” wherein all of their sides are completed sides and all of the Si atoms are completely saturated.

The EPOSS molecules of the present invention are distinguishable from other Polyhedral Oligomeric SilSesquioxanes that can exist in, for example, the ladder configuration of Formula IV, as well as those which have a core containing 6, 7 or 8 Si atoms. At least four of the Si atoms are bound, through an oxygen atom, to at least three of the other Si atoms (referred to herein as being “completely saturated”). All of the Si atoms are bound to at least one other Si atom through an oxygen atom. EPOSS in accordance with one aspect of the present invention are rigid three-dimensional cage-like structures in another embodiment, they have at least two completed sides. This rigid cage-like structure is distinguished from ladders and other structures which are not held in place in three directions. At least one, often eight or more, and more often all of the Si atoms in the cage-like structure are bound to at least one R group other than H. Preferably no more than three Si atoms are bound to more than two R groups. In another embodiment, no more than two Si atoms are bound to more than two R groups. In another embodiment, no more than one Si atom is substituted with more than two R groups.

The groups used for substituents on the core EPOSS cage, the “R groups” of the present invention include, without limitation, groups that impart conditioning to the hair, scalp and/or skin, groups including cationic species such as a quaternary nitrogen or “quat” as well as groups including a silicone and zwitterionic compounds. Groups that impart protection from solar radiation including UVA, UVB and/or UVC may also be substituted onto the EPOSS structure as can oxidative dyes, fluoroalkyl groups, amino groups and the like. These substituent groups, UV protecting, conditioning or otherwise, may be bound directly to the cage structure or may be bound through a bridging molecule such as an azo, diazo, epoxy or halogen containing material. Of course, UV protecting compounds, quats and other ingredients useful in cosmetic and personal care may also be mixed with EPOSS molecules as well. Some of these EPOSS derivatives, when used in correct amounts and formulated properly, can impart desirable properties to keratinous materials and/or to cosmetic and personal care formulations. Without limitation, depending upon EPOSS and the formulation, they may be effective in conditioning, dying, viscosity modification, film forming, UV protection, emulsification, enhancing substantivity, providing strength, compatibilize and the like. In addition, the use of EPOSS containing molecules in accordance with the present invention can often enhance the performance of other traditional ingredients used in such formulations in unexpected and superior ways. Stated another way, an improved UV protecting hair care compound may be obtained either by adding a EPOSS molecule exhibiting UV protecting properties or by adding EPOSS with no UV protecting properties to a UV protecting formulation to improve other properties such as substantivity, compatibility, etc.

Substituted EPOSS molecules including, without limitation, those described immediately above may also be used as monomers or as side groups in the creation of polymers. Some of these polymers can provide desirable properties such as film forming properties, production of tough yet breathable coatings, even conditioning. Indeed, the inclusion of a substituted EPOSS molecule as part of the polymer backbone or as a pendent group can, in some instances, provide not only the expected beneficial properties of polymers, but also additional advantages. For example, a polymer may be rendered tough and yet maintain good conditioning properties through the creation of a polymer with, for example, acrylic EPOSS polymers derivatized with conditioning quat groups.

In other particularly preferred aspects of the present invention, EPOSS molecules are provided, alone or as part of a polymer, which are multifunctional. For example, a EPOSS molecule may be substituted in one or more positions with a group capable of providing UV protection and also with a group capable of providing conditioning. This same EPOSS molecule could be incorporated into the backbone of a polymer as a monomer or can be attached to the backbone of a polymer as a pendent group.

In addition, as EPOSS molecules may have no less than nine Si atoms and many active sites susceptible to substitution, EPOSS molecules may provide a much greater density of active substituents. This plurality of possible substitutions can provide particularly desirable properties. For example, a EPOSS molecule could be substituted in eight or more positions with a cationic quat containing substituent. Not only might such a molecule provide excellent conditioning, it might also provide better substantivity. Indeed, in an embodiment, such an EPOSS molecule, because of its ability to essentially link to different hair strands (although not necessarily chemically or covalently, e.g., physically), may also be useful in providing styling products such as gels and mousses with excellent conditioning properties. Indeed, in one embodiment, EPOSS molecules can be used as additives with the hair styling technology described in U.S. Pat. No. 6,548,051 and U.S. patent Publication No. US 2002/0059941.

In an embodiment, the present invention provides EPOSS molecules including one or more of the following groups: hydrogen, methyl, ethyl, propyl, isobutyl, isooctyl, phenyl, cyclohexyl, cyclopentyl, —OSi(CH₃)₂—CH₂—CH₂—(CF₂)₅CF₃, —(CH₂)₃SH, N⁺(CH₃)₃, O⁻ N⁺(CH₃)₃, —OH, —(CH₂)ₙN⁺H₃X⁻ wherein n is 0-30 and X is a counter ion,

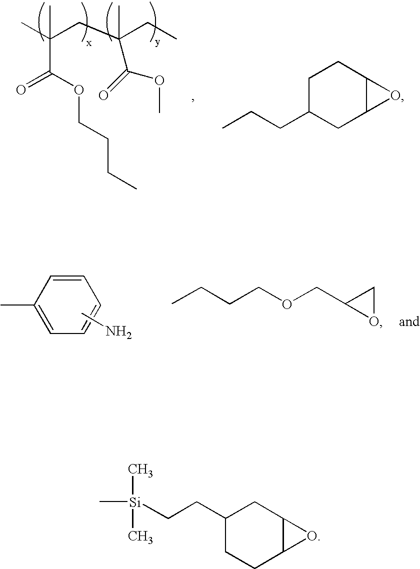

In one embodiment of the present invention, there is provided a personal care product comprising: at least one EPOSS having a rigid three-dimensional cage (or cage-like) structure comprising nine or more Si atoms within its cage structure. The EPOSS is present in an amount effective to provide a desired degree of a predetermined personal care property. Also included is at least one personal care ingredient. Personal care products include, without limitation, a shampoo, a nail polish, a conditioner or a mascara. Personal care products including the EPOSS molecules described herein generally, as well as those including the groups described immediately above, are all contemplated. These personal care products generally include an amount of EPOSS which is effective to provide a desired degree of a predetermined personal care property. The balance of these products is generally one or more personal care ingredients. In other embodiments, the present invention relates to nail polish and hair care products (e.g., lotions, shampoos, conditioners, styling gels, hair sprays, waxes and styling mousses) that include EPOSS molecules as recited herein. Methods of applying EPOSS containing personal care products and methods of using EPOSS containing personal care products to treat keratinous materials and/or to diminish the visible signs of aging, to protect skin or hair from damage from the sun and environment, and the like are also contemplated.

In one aspect of the invention there is provided a personal care product comprising at least one EPOSS. The EPOSS has a rigid three dimensional cage structure comprising nine or more Si atoms within its cage structure. In one embodiment, the EPOSS includes at least three, and preferably four of the Si atoms are completely saturated and all of the Si atoms are bound to at least one other of the Si atoms within the cage structure through an oxygen atom. The rigid three dimensional cage structure often has at least two completed sides and, in one embodiment, each of the Si atoms is bound to at least one R group. The EPOSS is present in an amount effective to provide a desired degree of a predetermined personal care property. The personal care product also includes at least one personal care ingredient. These personal care products may be formulated as a sunscreen composition, suntan product, antiperspirant, deodorant, cold cream, moisturizer, cleaner, shampoo, conditioner, dual shampoo/conditioner, rinse, cream rinse, cosmetic, hair coloring, hair dye, bleaching composition, styling product, cleansing cream, soap, perfume and cologne, powder, permanent waving product, relaxers, preshave, shaving cream, shaving product, after shaving product, bath product, self tanning product, bleaching product, and hair shine product. These personal care products may be provided in the form of a liquid, solid, cream, ointment, solution, gel, mousse, stick, cream, spray, powder, emulsion or dispersion.

### DETAILED DESCRIPTION

The term “personal care product(s)” in accordance with the present invention is meant broadly and encompasses products used for skin and hair and for their care, treatment or protection, as well as products for other cosmetic purposes. Without limitation, personal care products in accordance with the present invention include: sunscreen compositions, suntan products, antiperspirant and deodorant compositions, cold creams, moisturizers, cleaners, shampoos, conditioners (both conditioners that remain on the skin/hair and those that are rinsed), dual shampoo/conditioners, rinses, cream rinses, cosmetics (a term which includes, without limitation, lipsticks, lip balms, liners, mascara, blush, foundation, rouge, nail polish, nail enamel, nail varnish, nail conditioners, nail dyes, (collectively “nail polish” as the context will permit)), hair coloring, hair dyes, bleaching compositions, styling products (gels, mousses, sticks, creams, sprays), cleansing creams, soaps, perfumes and colognes, powders, permanent waving products, relaxers, preshaves, shaving creams and shaving products, after shaving products, bath products, self tanning products, bleaching products, and hair shine products.

Generally, EPOSS molecules in accordance with one of embodiment of the present invention have the cage or cage-like structure of Formula Ia-e (collectively Formula I).

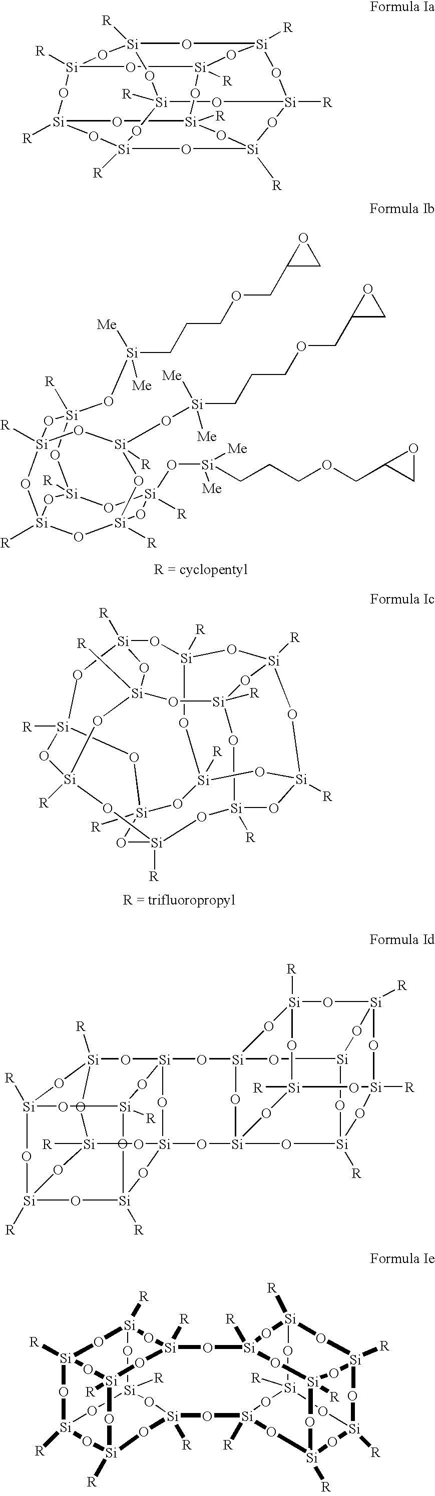

In Formula Ia, the number of Si atoms in the cage is 10, in Formula Ib, the number of Si atoms is 10 and in Formula Ic, the number of Si atoms in the cage is 12. In Formulae Id and Ie, the number of Si atoms in the cage or core is 16. It is also possible that one or even two of the oxygen bridges between successive silicon atoms are broken or missing. By way of non-limiting examples, consider the rigid three-dimensional cage structure illustrated in Formula II:

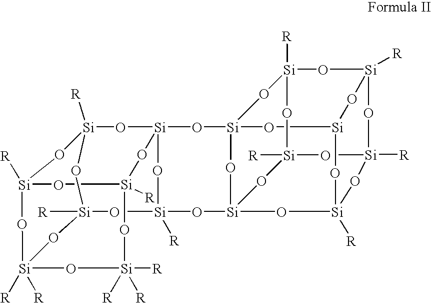

Consider also Formula IV:

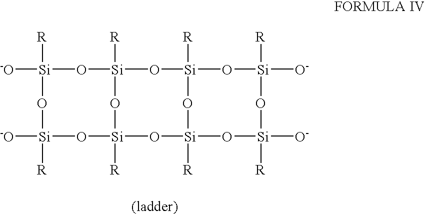

Formula IV, a ladder configuration (not an POSS according to the invention), can be a monomer linked end to end to other similar structures. It is not rigid within the meaning of this document as it can fold or flex around each R—Si—O—Si—R axis of the molecule. No such movement is possible in the rigid 3-D cage structures (whether complete or incomplete) of the invention. Thus, the molecules of this formula are not EPOSS.

Note also that when referencing EPOSS molecules as being in accordance with Formula II or just having the structure of Formula II, the sides which are “open,” missing or broken are illustrative only. When reference is made to Formula II, it is understood that any one or two sides, or any one or two oxygen bridges, may be broken or missing. The structure of the EPOSS molecule can be roughly thought of as a many sided box or cage in shape with silicon (Si) atoms at each corner. Each Si atom is connected to at least one other Si atom through bonds to an oxygen atom (also referred to as an “oxygen bridge”). Preferably, at least four of the Si atoms in the EPOSS structure are “completely saturated.” Most preferably, most if not all of the Si atoms are bound, through oxygen atoms, to three other Si atoms within the cage, and thus are “completely saturated.” While illustrated as Si atoms, the groups at each corner may be the same or different and may be one or more atoms or groups including, without limitation, silicon, silane, siloxane, silicone or organometallic groups. The number of Si atoms will range from 9 to 100, alternatively 9 to 30, also alternatively 9 to 20 and finally alternatively 9 to 16, and all of the Si atoms of the cage or core are bound to at least one other Si atom through an oxygen bridge. The EPOSS of the invention also preferably exists in a rigid 3-dimensional cage structure as illustrated, for example, in Formula I and the cage may, in one embodiment, have at least two completed sides (all Si atoms for that side being completely saturated). Each Si is preferably bound to at least one R group.

The one remaining bond of each silicon (sometimes more) can bind to a variety of substituents or groups specified, as “R” groups. It is possible to eliminate as many as three such bridges to a particular Si atom. As stated above, the Si molecules not completely bound as described previously may have one or more additional positions available for binding additional substituents.

EPOSS molecules may in some cases have a distance between the Si groups of about 0.5 nanometers. The distance from the end of attached R groups will depend largely on the choice of those groups. Generally, the distance is about 1.5 nanometers.

EPOSS compounds with various R groups are well-known in the literature. They are described in a number of patents including, without limitation, Weidner et al., U.S. Pat. No. 5,047,492, issued Sep. 10, 1991, Sojka, U.S. Pat. No. 5,389,726, issued Feb. 14, 1995, Lichtenhan et al., U.S. Pat. No. 5,484,867, issued Jan. 16, 1996, Lichtenhan et al., U.S. Pat. No. 5,589,562, issued Dec. 31, 1996, Crocker et al., U.S. Pat. No. 5,750,741, issued May 12, 1998, Banaszak Holl et al., U.S. Pat. No. 5,858,544, issued Jan. 12, 1999, Lichtenhan et al., U.S. Pat. No. 5,939,576, issued Aug. 17, 1999, Lichtenhan et al., U.S. Pat. No. 5,942,638, issued Aug. 24, 1999, Lichtenhan et al., U.S. Pat. No. 6,100,417, issued Aug. 8, 2000, Van Santen et al., U.S. Pat. No. 6,127,557, issued Oct. 3, 2000, Takamuki et al., U.S. Pat. No. 6,207,364, issued Mar. 27, 2001, Zank et al., U.S. Pat. No. 6,252,030, issued Jun. 26, 2001, Nguyen, U.S. Pat. No. 6,270,561, issued Aug. 7, 2001, Mehl et al., U.S. Pat. No. 6,277,451, issued Aug. 21, 2001, Lichtenhan et al., U.S. Pat. No. 6,362,279, issued Mar. 26, 2002, and Barbee et al., U.S. Pat. No. 6,486,254, issued Nov. 26, 2002. These patents describe in detail various methods of producing the basic EPOSS cage structure and various derivatives thereof, including EPOSS based polymers. To the extent that these patents identify and describe various EPOSS molecules including at least nine silicon atoms, derivatives and polymers thereof, they are incorporated by reference. The discussions of techniques for manufacturing and derivatizing this class of compounds described in each of these patents is also hereby incorporated by reference.

In general, R groups can be the same or different and may be reactive or nonreactive groups. They may be, individually H, hydroxy (—OH), alkanes derivatives (missing a hydrogen) also known as alkyl groups, alkenyl groups, also referred to as derivatives of alkenes (having one or more double bonds), usually missing an H where they are bound to Si in EPOSS or to some other molecule, alkynyl groups also referred to as derivatives of alkynes (having one or more triple bonds) usually missing an H where they are bound to Si in EPOSS or to some other molecule, aryl groups (either the 6-carbon ring of benzene or the condensed 6-carbon rings of other aromatic derivatives such as naphthalene) also referred to as derivatives of arenes, usually missing an H where they are bound to Si in EPOSS or to some other molecule, acyl groups (organic acids without the OH group, e.g., CH₃CO— or C₆H₅CO—), alkoxy groups (alkyl radicals attached to the remainder of a molecule by oxygen), such as methoxy, ester groups, acid groups, acrylate groups, alkyl acrylate groups, hydroxy groups, halogens, amino groups, alkylamino groups, aminoalkyl groups, groups containing one or more tertiary or quaternary nitrogens, silicone containing groups, sulfur containing groups, epoxides, azo groups, diazo groups, halogens, cyclic compounds which can undergo ring opening polymerization or ring opening metathesis polymerization. R groups may also be monomers or polymers where EPOSS will be used as a pendant substituent of the polymer. Acrylates and cationic polymers providing conditioning properties are provided in one embodiment.

Where appropriate, any of these R groups may themselves be substituted or unsubstituted, saturated or unsaturated, linear or branched. Possible substitutions include C₁-C₃₀ alkyl groups, C₁-C₃₀ alkenyl groups, C₁-C₃₀ alkynyl groups, C₆-C₁₈ aryl groups, acyl groups, alkoxy groups, carboxy groups, ester groups, acrylate groups, alkyl acrylate groups, trihydroxy groups, amino groups, alkylamino groups including mono and dialkylamino groups, mono and dihydroxy alkylamino groups, cyano groups, aminoalkyl groups, groups containing one or more tertiary or quaternary nitrogens, silicone containing groups, sulfur and/or phosphorous containing groups, SO₂X, SO₃X, where X is H, methyl or ethyl, epoxides, azo groups, diazo groups, halogens, cyclic compounds which can undergo ring opening polymerization or ring opening metathesis polymerization. Indeed, any group which can be attached to a corner of a EPOSS molecule can be used.

When these R groups include carbon containing fatty acids or fatty alcohols, aromatic or cyclic groups, they generally may contain between six and 50 carbon atoms and may be saturated or unsaturated, substituted as discussed above or unsubstituted and branched or linear, as appropriate for a given group.

More specifically, possible R groups include, without limitation, hydroxy groups including mono or poly hydroxy groups, phenols, alkoxy, hydroxy alkyls, silanes, amino and in particular, quats, halosilanes, epoxides, alkyl carbonyls, alkanes, haloalkyls, halogens, acrylates, methacrylates, thiols, nitriles, norbornenyls, branched alkyl groups, polymers, silanes, silanols, styryls and thiols. In a single representative EPOSS molecule of nine Si atoms, the R groups may include H, —OH, —NH2, —CH₂CH₂N+CH₃ (OCH₂CH₃) CH₂CH₂CH₃, —CH₂CH₂CHOCH₂(epoxide), —OC(CH₃)₃, —OOC(CH₂)₁₆CH₃ and CH₃ could be Cl. This is a hypothetical example, merely to illustrate that each of the R groups can be derivatized separately and to emphasize the wide variety of possible substitutions. Other R groups include portions of other molecules often used as personal care ingredients, such as UV-protecting groups derived from UV-protecting compounds.

In one embodiment, these EPOSS molecules are not completely substituted with the same R groups (e.g., not all methyl).

Another embodiment of the present invention provides EPOSS molecules including one or more of the following R groups: H, methyl, ethyl, propyl, isobutyl, isooctyl, phenyl, cyclohexyl, cyclopentyl, —OSi(CH₃)₂—CH₂—CH₂—(CF₂)₅CF₃, —(CH₂)₃SH, N⁺(CH₃)₃, O⁻ N⁺(CH₃)₃, —OH, —(CH₂)ₙN⁺H₃X⁻ wherein n is 0-30 and X is a counter ion,

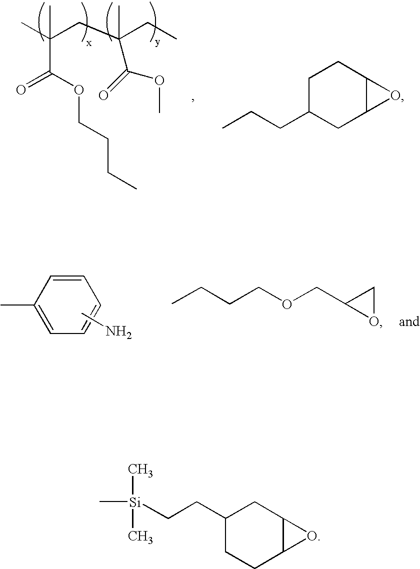

Also contemplated under the term EPOSS is the family of commercially available compounds available from Hybrid Plastics, 18237 Mount Baldy Circle, Fountain Valley, Calif. 92708-6117 and Mayaterials, Inc. P.O. Box 87, South Lyon, Mich. 48178-0087.

EPOSS molecules available from Hybrid Plastics include, without limitation, those having nine or more Si atoms which are selected from alcohols and phenols such as (TMP DiolCyclopentyl-POSS, TMP DiolIsobutyl-POSS, Trans-CyclohexaneDiolCycohexyl-POSS, Trans-CyclohexaneDiolIsobutyl-POSS, 1,2-PropaneDiolCyclohexyl-POSS, 1,2-PropaneDiolIsobutyl-POSS, and OctaHydroxypropyldimethylsilyl-POSS), alkoxysilanes such as (DiethoxymethylsilylethylCycohexyl-POSS, DiethoxymethylsilylethylIsobutyl-POSS, DiethoxymethylsilylpropylCyclohexyl-POSS, DiethoxymethylsilylpropylIsobutyl-POSS, EthoxydimethylsilylethylCyclohexyl-POSS, EthoxydimethylsilylethylIsobutyl-POSS, EthoxydimethylsilylpropylCyclohexyl-POSS (may contain some isomer), EthoxydimethylsilylpropylIsobutyl-POSS (may contain some β-isomer), TriethoxysilylethylCyclohexyl-POSS, TriethoxysilylethylIsobutyl-POSS, TriethoxysilylpropylCyclohexyl-POSS (may contain some isomer) and TriethyoxysilylpropylIsobutyl-POSS (may contain some β-isomer), amines such as (AminopropylCyclohexyl-POSS, AminopropylIsobutyl-POSS, AminopropylIsooctyl-POSS, AminoethylaminopropylCyclohexyl-POSS, AminoethylaminopropylIsobutyl-POSS, OctaAminophenyl-POSS and OctaAmmonium-POSS), chlorosilanes such as (MonoChloroCyclohexyl-POSS, MonoChloroCyclopentyl-POSS, MonoChloroIsobutyl-POSS, ChlorodimethylsilylethylCyclohexyl-POSS, ChlorodimethylsilylethylIsobutyl-POSS, ChlorodimethylsilylpropylCyclohexyl-POSS (may contain some β-isomer), chlorodimethylsilylpropylIsobutyl-POSS (may contain some β-isomer), DichloromethylsilylethylCyclohexyl-POSS, DichloromethylsilylethylIsobutyl-POSS, DichloromethylsilylpropylCyclohexyl-POSS (may contain some isomer), DichloromethylsilylpropylIsobutyl-POSS (may contain some β-isomer), TrichlorosilylethylCyclohexyl-POSS, TrichlorosilylethylIsobutyl-POSS, TrichlorosilylpropylCyclohexyl-POSS (may contain some β-isomer), TrichlorosilylpropylIsobutyl-POSS (may contain some β-isomer), Octa(chlorosilylethyl)-POSS (may contain some β-isomer), Octa(dichlorosilylethyl)-POSS (may contain some β-isomer) and Octa(trichlorosilylethyl)-POSS (may contain some β-isomer), epoxides such as (EpoxyCyclohexylCyclohexyl-POSS, EpoxyCyclohexylCyclopentyl-POSS, EpoxyCyclohexylIsobutyl-POSS, EpoxyCyclohexylDisilanolIsobutyl-POSS, EpoxyCyclohexyl-POSS Cage Mixtures such as EpoxypropylCyclopentyl-POSS, EpoxypropylIsobutyl-POSS, GlycidylCyclohexyl-POSS, GlycidylCyclopentyl-POSS, GlycidylEthyl-POSS, GlycidylIsobutyl-POSS, GlycidylIsoctyl-POSS, GlycidylPhenyl-POSS, OctaEpoxyCyclohexyldimethylsilyl-POSS, OctaGlycidyldimethylsilyl-POSS, TrisGlycidylCyclohexyl-POSS, TrisGlycidylCyclopentyl-POSS, TrisGlycidylEthyl-POSS and TrisGlycidylIsobutyl-POSS), esters such as (EthylUndecanoateCyclohexyl-POSS, EthylUndecanoateCyclopentyl-POSS, EthylUndecanoateIsobutyl-POSS, MethylPropionateCyclohexyl-POSS, MethylPropionateCyclopentyl-POSS and MethylPropionateIsobutyl-POSS), fluoroalkyls such as (Fluoro(3)DisilanolCyclopentyl-POSS, Fluoro(13)DisilanolCyclopentyl-POSS, Fluoro(13)DisilanolIsobutyl-POSS, MethacrylFluoro(3)Cyclopentyl-POSS mixture of isomers, MethacrylFluoro(13)Cyclopentyl-POSS mixture of isomers, MethacrylFluoro(3)Isobutyl-POSS mixture of isomers, DodecaTrifluoropropyl-POSS, TriFluoroCyclohexyl-POSS, TriFluoroCyclopentyl-POSS, TriFluoroIsobutyl-POSS, TrifluoropropylIsobutyl-POSS, TrisFluoro(3)Cyclopentyl-POSS and TrisFluoro(13)Cyvlopentyl-POSS), halides such as (ChlorobenzylCyclohexyl-POSS, ChlorobenzylCyclopentyl-POSS, ChlorobenzylIsobutyl-POSS, ChlorobenzylethylCyclohexyl-POSS, ChlorobenzylethylCyclopentyl-POSS, ChlorobenzylethylIsobutyl-POSS, ChlorophenylCyclohexyl-POSS, ChlorophenylCyclopentyl-POSS, ChlorophenylIsobutyl-POSS, ChlorophenylPhenyl-POSS, ChloropropylCyclohexyl-POSS, ChloropropylCyclopentyl-POSS and ChloropropylIsobutyl-POSS), isocyanates such as (IsocyanatopropyldimethylsilylCyclohexyl-POSS and IsocyanatopropyldimethylsilylIsobutyl-POSS), methacrylates & acrylates such as (AcryloCyclohexyl-POSS, AcryloCyclopentyl-POSS, AcryloIsobutyl-POSS, MethacrylCyclohexyl-POSS, MethacrylCyclopentyl-POSS, MethacrylEthyl-POSS, MethacrylIsobutyl-POSS, MethacrylIsooctyl-POSS, 90%, MethacrylPhenyl-POSS, MethacrylDisilanolCyclohexyl-POSS, MethacrylDisilanolCyclopentyl-POSS, MethacrylDisilanolIsobutyl-POSS, MethacrylFluoro(3)Cyclopentyl-POSS, MethacrylFluoro(13)Cyclopentyl-POSS, MethacryltrimethylsiloxyCyclopentyl-POSS, MethacryltrimethylsiloxyIsobutyl-POSS, Methacryl-POSS Cage Mixture, OctaMethacryldimethylsilyl-POSS, TrisMethacrylCyclohexyl-POSS and TrisMethacrylIsobutyl-POSS), molecular silica such as (DodecaPhenyl-POSS, DodecaPhenyl-POSS, 85%, Isooctyl-POSS Cage Mixture, 95%, OctaCyclohexyl-POSS, OctaCyclopentyl-POSS, OctaIsobutyl-POSS, OctaMethyl-POSS, OctaPhenyl-POSS, OctaTMA-POSS, DodecaTrifluoropropyl-POSS, OctaTrimethylsiloxy-POSS, Phenethyl-POSS Cage Mixture and PhenethylIsobutyl-POSS), nitriles such as (CyanoethylCyclohexyl-POSS, CyanoethylCyclopentyl-POSS, CyanoethylIsobutyl-POSS, CyanopropylCyclohexyl-POSS, CyanopropylCyclopentyl-POSS and CyanopropylIsobutyl-POSS), norbornenyls such as (NorbornenylethylCyclohexyl-POSS, NorbornenylethylIsobutyl-POSS, NorbornenylethylDiSilanolCyclohexyl-POSS, NorbornenylethylDiSilanolCyclopentyl-POSS, NorbornenylethylDiSilanolIsobutyl-POSS, Tris NorbornenylCyclohexyl-POSS, Tris NorbornenylCyclopentyl-POSS and Tris NorbornenylIsobutyl-POSS), olefins such as (AllylCyclohexyl-POSS, AllylCyclopentyl-POSS, Allylsobutyl-POSS, AllylDimethylsilylCyclopentyl-POSS, CyclohexenylethylCyclopentyl-POSS, DimethylvinylCyclopentyl-POSS, DiphenylvinylCyclopentyl-POSS, MonoVinylCyclohexyl-POSS, MonoVinylCyclopentyl-POSS, MonoVinylIsobutyl-POSS, PhenylMethylVinylCyclopentyl-POSS, Tris(Dimethylvinyl)Isobutyl-POSS, TrivinylsilylCyclopentyl-POSS, OctaCyclohexenyldimethylsilyl-POSS, OctaVinyldimethylsilyl-POSS, OctaVinyl-POSS and Vinyl-POSS Cage Mixture), phosphines such as (DiphenylphosphinoethylCyclopentyl-POSS and DiphenylphosphinopropylCyclopentyl-POSS), polymers such as (Poly(dimethyl-co-methylhydrido-co-methylpropylPOSS)siloxane, Poly(dimethyl-co-methylvinyl-co-methylethylsiloxyPOSS)siloxane, OctaMethyl-POSS Nanoreinforced™ Polypropylene, lowt %, Poly(ethylsilsesquixane) uncured, Poly(methylsilsesquioxane) uncured, Poly(phenylsilsesquioxane) uncured, Poly(propylmethacrylPOSS-co-methylmethacrylate), Poly(propylmethacrylPOSS-co-styrene), Poly(styrylPOSS-co-styrene), Poly(vinylsilsesquioxane) uncured and Poly(vinylsilsesquioxane) fully cured FireQuench™), silanes such as (DimethylsilaneCyclohexyl-POSS, DimethylsilaneCyclopentyl-POSS Schwab Hydride, DimethylsilaneIsobutyl-POSS, MonoSilaneCyclohexyl-POSS, MonoSilaneIsobutyl-POSS, OctaSilane-POSS, Tris(Dimethylsilane)Cyclohexyl-POSS, Tris(Dimethylsilane)Cyclopentyl-POSS and Tris(Dimethylsilane)CycloIsobutyl-POSS), silanols such as (CyclohexenyldimethylsilyDisilanolIsobutyl-POSS, DimethylphenylDisilanolCyclopentyl-POSS, DimethylvinylDisilanolCyclohexyl-POSS, DimethylvinylDisilanolCyclopentyl-POSS, DimethylvinylDisilanolIsobutyl-POSS, DiSilanolCyclopentyl-POSS, DiSilanolIsobutyl-POSS, EpoxyCyclohexylDisilanolIsobutyl-POSS, Fluoro(3)DisilanolCyclopentyl-POSS, Fluoro(13)DisilanolCyclopentyl-POSS, Fluoro(13)DisilanolIsobutyl-POSS, MethacrylDisilanolCyclohexyl-POSS, MethacrylDisilanolCyclopentyl-POSS, MethacrylDisilanolIsobutyl-POSS, MonoSilanolCyclohexyl-POSS, MonoSilanolCyclopentyl-POSS Schwabinol, MonoSilanolIsobutyl, NorbornenylethylDiSilanolCyclohexyl-POSS, NorbornenylethylDiSilanolCyclopentyl-POSS, NorbornenylethylDiSilanolIsobutyl-POSS, TMS DiSilanolCyclohexyl-POSS, TMS DiSilanolIsobutyl-POSS, TriSilanolCyclohexyl-POSS, TriSilanolCyclopentyl-POSS, TirSilanolEthyl-POSS, TriSilanolIsobutyl-POSS, TriSilanolIsooctyl-POSS and TriSilanolPhenyl-POSS), styrenes such as (StyrenylIsobutyl-POSS, StyrylCyclohexyl-POSS, StyrylCyclopentyl-POSS and StyrylIsobutyl-POSS), and thiols such as (MercaptopropylCyclohexyl-POSS, Mercaptopropylsobutyl-POSS and MercaptopropylIsooctyl-POSS, 90%). Other EPOSS products may be purchased from ALDRICH. Still others are described in Weidner et al. U.S. Pat. No. 5,047,492 issued Sep. 10, 1991, the text of which, and in particular, the POSS molecules described in the passage of column 1, line 22 through column 2, line 48, are hereby incorporated by reference and Barry et al. U.S. Pat. No. 2,465,188, issued Mar. 27, 1948, the text of which is also hereby incorporated by reference. See also Banaszak Holl et al., U.S. Pat. No. 5,858,544, issued Jan. 12, 1989, the text of which is also incorporated by reference.

Mixtures of EPOSS molecules are specifically contemplated as are mixtures of one or more EPOSS molecules with other silicone resins. When part of a mixture with other silicone resins, it is possible that at least one EPOSS molecule or a mixture of EPOSS molecules as described herein may be present in an amount at least equal to the amount of such other silicone resins in the mixure. This is particularly true where one of the other silicone resins is in a ladder configuration. Mixtures of EPOSS molecules with nonextended Polyhedral oligomeric silsesquioxanes (“POSS”), molecules containing between six and eight Si atoms within their cage-like structure are also contemplated. POSS molecules are also available commercially from Hybrid Plastics.

As previously noted and as reflected in the patents and publications previously incorporated by reference, there are many known EPOSS molecules and many known ways to produce EPOSS compounds and various derivatives and polymers therefrom. In general, however, one process of producing EPOSS includes the following steps: a) providing a trifunctional polyhedral oligomeric silsesquioxane of the formula Si₇R₇₀ ₉(OA)₃, where OA is —OH, —OSb(CH₃)₄, —OSn(CH₃)₃, or —OTl, and R is an alkyl, alkenyl, aryl, alkoxy group or other R group described herein; and b) corner capping said trifunctional polyhedral silsesquioxane by reacting said trifunctional polyhedral silsequioxane with a compound of the formula M-Z to form a polyhedral oligomeric silsesquioxane having the formula Si₇R₇₀ ₁₂M(Z). M is a silane, siloxane or organometallic group and Z is a reactive group selected from the group consisting of chloride, bromide or iodide. The process further includes the step of adding silver perchlorate to a solution of the polyhedral oligomeric silsesquioxane in aqueous acetone to convert reactive group Z to an alcohol. See U.S. Pat. No. 5,484,867. POSS molecules may also be made as described in a paper entitled “Polyhedral Oligosilsesquioxanes and Heterosilsesquioxanes” by Frank J. Feher of the Department of Chemistry of the University of California at Irvine, Calif. 92697-2025 USA available from Gelest, Inc., the test of which is hereby incorporated by reference.

EPOSS molecules in accordance with the present invention include those substituted with one or more cation containing groups. These cationic groups are generally useful in conditioners and hair care products, although, particularly when more than one R group is substituted with a cation containing species, they may also be used as styling agents as ionic attractive forces can exist between the different cationic species and negatively charged or anionic sites on more than one strand of hair, for example.

Polycationic quats may be useful in styling and holding products such as sprays, gels and mousses. The cation containing groups need not be cationic in all environments or at all times. For example, some groups may only be cationic at certain pH or at certain concentrations. In addition, the POSS molecule may contain a group that includes a tertiary nitrogen, which after its attachment to the POSS structure is later quaternized. Any cationic species useful in personal care products is contemplated, which include groups containing Si or N, the latter of which includes quats. Quat groups include the quat monomers containing one or more quaternary ammonium groups described in Cannell et al., U.S. Pat. No. 6,486,105, the text of column 7, line 66 through column 7, line 47 is hereby incorporated by reference.

As shown in Formula Va, a molecule used for illustration only, an EPOSS molecule has been substituted such that one of the R groups is a benzyl chloride group to form a chlorobenzyl EPOSS. The remaining R groups may be, for example, hydrogen, methyl, isobutyl, cyclohexyl or cyclopentyl groups.

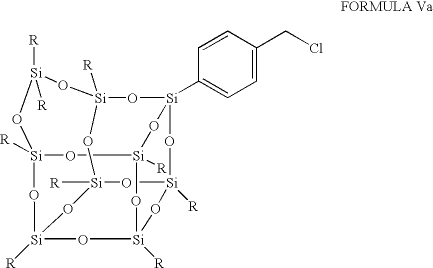

This EPOSS molecule is reacted with triethylamine (TEA) to produce the quat molecule illustrated in Formula Vb.

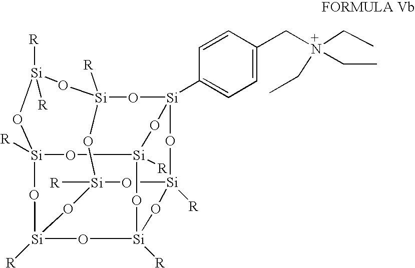

Note that in Figure Vb, the quat is not and need not be attached directly to the silicon of the EPOSS molecule. Instead, a bridging molecule is used. In Formula Va, the bridging molecule is benzyl chloride. However, any molecule that be used to connect another group, including cationic groups, to a EPOSS molecule is contemplated. This can include, without limitation, compounds like epichlorhydrin, which includes both a reactive epoxy group and a chloride leaving group and can be used to connect a group to a EPOSS molecule and/or to quaternize a tertiary nitrogen. Any molecule that can be used to attach a specified group to a EPOSS molecule at one of the silicon atoms is contemplated. These include halogenated groups such as alkyl halides, aryl halides, and epoxy groups. Bridging molecules may first be derivatized to a EPOSS molecule and then substituents added thereto or the reverse. By using different bridging molecules with different affinities, one can often direct the specific binding of different substituents on the EPOSS molecule. Certain epoxy containing bridges, for example, may have a greater affinity for certain substituents while certain alkyl halide containing groups may be more easily reacted with others. It is possible, therefore, to in some way direct the position and/or number of two or more groups to be attached to each EPOSS nucleus.

These EPOSS compounds can also be used as monomers for the creation of polymers. Again, there are many ways of accomplishing polymerization of EPOSS molecules. However, in general, the process can include the steps of providing a polyhedral oligomeric silsesquioxane of the formula Si₇R₇O₁₂M(Z), where R is an alkyl, alkenyl, aryl, or alkoxy group, or one of the other R groups discussed herein, M is a silane, siloxane or organometallic group, and Z is a reactive group that includes, without limitation, hydrides, esters, acids, acrylates, alcohols, amines, alkoxides, aldehydes, halides, halogen-substituted alkyl groups, halogen-substituted alkenyl groups, halogen-substituted aryl groups, alpha-epoxides, and cyclic compounds which can undergo a ring opening polymerization or ring opening metathesis polymerization; and reacting the polyhedral oligomeric silsesquioxane at the site of the reactive Z group with an oligomer, polymer, reactive co-monomer, or catalyst to form a homopolymer or copolymer containing silsesquioxane groups.

Alternatively, EPOSS molecules can be derivatized to a polymer backbone such as an acrylate or methacrylate to modify the properties of the polymer and to add functionality. Various acrylates and methacrylates are used, for example, in nail polish. The addition of various EPOSS molecules bound to, for example, methacrylates can improve the film forming properties of existing acrylate and methacrylate polymers and can add additional conditioning and/or UV protection, depending upon how the POSS molecule is substituted.

This latter type of polymer is exemplified by the polymer illustrated below in Formula VIa.

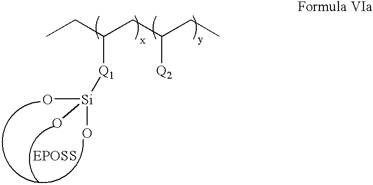

The R groups for the EPOSS molecules may be H, methyl, hydroxy, isobutyl, cyclopentyl, cyclohexyl and phenyl groups. They can also be any of the other groups identified previously for R groups as well. Alternatively, a chlorobenzyl functionalized EPOSS may be quaternized using a dialkyl amino alkyl methacrylate, acrylate or acrylamide including, for example, dimethyl amino propyl methacrylate, to form the quat. This could be then used as a monomer to make an acrylic polymer having pendent quaternary EPOSS groups. Of course, any EPOSS structure can be used as a monomer or be attached to a polymer backbone.

EPOSS molecules can be bound as pendant groups to cationic polymers that can result in durable coatings, useful films and conditioning properties. These conditioning polymers include, without limitation, those described in U.S. Pat. No. 6,338,842 and in particular the text at column 4, line 62 through column 10, line 67, the text of which is hereby incorporated by reference. The polymer backbone from which EPOSS groups may be appended can be any homopolymer, copolymer or block copolymer which can be functionalized to attach an EPOSS. EPOSS may be bound directly or through a bridging molecule as previously described. By way of a non-limiting example, as shown in Formula V1, Q₁ and Q₂ may be the same or different and may be linking group or any R group which can act to or be derivatized to connect the cage structure to the polymer backbone. An example used for illustration only is shown in Formula VIb:

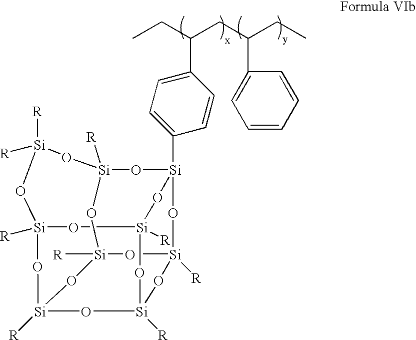

Q₁ and Q₂ are benzyl rings in this instance. However, they can also be, for example —COOR groups such as —COO(CH₂)₄— or —COO(CH₂)—. “X” in the case of EPOSS containing polymers ranges from between about 0.05 and about 100 mole percent and “Y” ranges from between 0 and about 99.95 mole percent thereof. The average molecular weight (weight average molecular weight) of such polymers is not critical so long as the resulting polymer can be applied and used in personal care products. Generally, however, the average molecular weight of the polymers will be about 1,500,000 or less, often 1,000,000 or less and even more often 500,000 or less.

EPOSS molecules may also be produced using azo groups and diazo groups as substituents. Indeed, EPOSS dimers could be produced linked by an azo group. The use of thermal energy or UV light can produce free radicals, allowing monomers and polymers to be added to the reactive nitrogen. In this way, the POSS molecule can act as an endcap. Chain propagation may be terminated by the use of another EPOSS containing molecule, depending on the selection of the monomer or polymer used, the result can be a structure that is hydrophobic at the ends and hydrophilic therebetween. Such materials may fold in on themselves forming interesting and unique structures with desirable film forming properties and utility as emulsifiers and viscosity modifiers. Similar results can be obtained by, for example, reacting polyethylene glycol with POSS molecules that have been derivatized with an epoxy group.

The amount of EPOSS used will vary. But usually, sufficient EPOSS is present in personal care products so as to a desired degree of at least one personal care property. A “predetermined personal care property” in accordance with the present invention is the reason why the EPOSS was added to a personal care product. This will vary with the product and with the intended role the EPOSS will play in that product. In a non-limiting example, a particular EPOSS compound may be added to a hair conditioner because it possesses desirable conditioning properties, either alone or when used in combination with another EPOSS molecule and/or one of the other components of the conditioner. Thus, the predetermined personal care property could be conditioning. EPOSS compounds could, however, play other roles in hair conditioners either instead of conditioning or in addition thereto. Without limitation, a EPOSS compound could be used as a thickener, a solubilizer or compatibilizer, a film form, coloring gel, a hair permeation enhancer, a substantivity enhancer, an adhesion promoter, a structuring agent, a UV protective agent and the like. Thus the reason for adding EPOSS molecules to a hair conditioner might or might not be conditioning.

An “amount effective” or an effective amount is an amount of one or more EPOSS compounds necessary to provide a “desired degree” of the predetermined personal care property. If the EPOSS was to be used, for instance, as the sole conditioning agent, then an effective amount of the EPOSS is that amount necessary to provide the desired degree of that predetermined personal care property. If the EPOSS was but one of a number of conditioners used, then the effective amount is an amount which provides, for instance, with the other conditioners, the desired degree of conditioning. If used as a thickener, the EPOSS is provided in an amount which is necessary to, for instance, alter thickness.

Preferably an “amount effective” or an effective amount to provide a desired degree of a personal care property is an amount that can provide at least some minimum, objectively measurable, and desirable change in that property when compared to the identical property in an otherwise identical personal care product formulated without that EPOSS.

Understandably, the amount of EPOSS used will vary widely with the EPOSS, the type of personal care product, the role of the EPOSS in that product, the use of additional EPOSSs, the type and amount of the other components used in the personal care product and other factors well-known to formulators. However, in general, the amount of EPOSS useful in accordance with the present invention is at least about 0.005% by weight of the final formulation. More particularly, the amount is at least about 0.01%. More particularly, the amount is at least about 0.05% and even more particularly at least about 0.10% by weight of the final formulation. The upper limit on the amount of POSS used is generally not critical, although a point of diminishing return is likely. As this will generally impact cost, but not performance, this is generally not an issue for formulation. However, the amount of EPOSS used will generally not exceed about 40%, more preferably about 20%, and even more preferably about 10%.

The personal care products of the invention also often include at least one “personal care ingredient.” This could be a single additional ingredient such as water, surfactant, pigment and the like. However, most frequently, personal care products are a mixture of personal care ingredients traditionally found in personal care products. In the case of a shampoo, again for example only, these personal care ingredients could include one or more of those found in L'Oreal Kids® Extra Gentle 2-in-1 Strawberry Smoothie Shampoo, including water, sodium laureth sulfate, PEG-200 hydrogenated glyceryl palmate, disodium cocoamphodiacetate, PEG-30 glyceryl cocate, glycol, distearate, PEG-7 glyceryl cocoate, hexylene glycol, disodium ricinoleamido MEA-sulfosuccinade, fragrance, sodium laureth-8 sulfate, polyquaternium-10, tartaric acid, sodium methylparaben, magnesium laureth-8 sulfate, magnesium laureth sulfate, DMDM hydantoin, sodium oleth sulfate, magnesium oleth sulfate, methylparaben and red color number 33. For a mascara, these personal care ingredients could include one or more of the components of Maybelline Great Lash® Very Black Mascara, which include water, beeswax, ozokerite, shellac, glyceryl stearate, triethanolamine, propylene glycol, stearic acid, sorbitan sesquioleate, methylparaben, quaternium-15, quaternium-22, simethicone, butylparaben, and iron oxides, titanium oxides and ultramarines. These personal care ingredients are used in their conventional amounts and generally comprise the balance of the personal care products of the present invention.

Personal care ingredients in accordance with the present invention include, without limitation, one or more absorbents, alphahydroxy acids, betahydroxy acids, polyhydroxy acids, antiacne agents, antiperspirants, anticaking agents, antifoaming agents, antimicrobial agents, antioxidants, antidandruff agents, astringents, binders, bleaching agents, buffers, biological additives, bulking agents, carriers, chelating agents, coupling agents, compatibilizers, conditioners, colorants, cosmetic astringents, cosmetic biocides, denaturants, drug astringents, detergents, deodorants, dispersants, external analgesics, emulsifiers, film formers, foaming agents, fragrances and fragrance components, hair styling ingredients, hair holding ingredients (mousses, sprays, etc.), hair conditioners, hair colors, hair growth promoters, humectants, keratolytics, moisturizers, straightening agents, oxidizers, mineral and organic particles, plastics, polymers, permanent waving agents, opacifying agents, perfumes, pH adjusters, pigments, preservatives, proteins, retinoids, reducing agents, sequesterants, skin bleaching agents, skin conditioning agents, skin smoothing agents, skin soothing agents, skin healing agents, softeners, solubilizing agents, surfactants, lubricants, thickeners, penetrants, permeation enhancers, analgesics, anti-inflammatory agents, antibiotics, anesthetics, plasticizers, salts, solvents essential oils, sunscreens and UV-absorbers, vitamins, provitamines, plant extracts, ceramides and pseudoceramides. The nature of the personal care ingredients used and their amounts will depend on the overall formulation, as well as its form, function and the keratinous material that is the target of its application. Understandably, the ingredients will also change depending on whether or not the product is a liquid, ointment, lotion, spray, gel, cream, liposome, microcapsule, emulsion, foam, paste, powder, granule, crystal, microcrystal or other solid. The ingredients may also depend on whether the preparations are aqueous or nonaqueous or are to be placed in a dispersion or a solution.

Personal care ingredients may also include, for example, keratin and its derivatives, melanin, collagen, cystine, chitosan and its derivatives, biotin, oligoelements, protein hydrolysates, and phospholipids. Other examples of personal care ingredients that may be mentioned are fatty alcohols, fatty acid esters of fatty alcohols, alkylated proteins, quaternized proteins, anionic, cationic, nonionic or amphoteric surfactants, silicones, volatile silicones, silicone oils, silicone gums, amino silicones, quaternized silicones, alkylated silicones, grafted silicones, silicone emulsions, mineral and plant oils, plant waxes, or pseudoceramides and polymers. Other examples of personal care ingredients include, without limitation, para-aminobenzoic acid (PABA), benzophenone-1, benzophenone-2, benzophenone-3, benzophenone-4, benzophenone-6, benzophenone-8, benzophenone-12, methoxycinnamate, ethyl dihydroxypropyl-PABA, glyceryl PABA, homosalate, methyl anthranilate, octocrylene, octyl dimethyl PABA, octyl methoxycinnamate, octyl salicylate, PABA, 2-phenylbenzimidazole-5-sulphonic acid, triethanolamine salicylate, 3-(4-methylbenzylidene)-camphor, avobenzone, and 2,6-dicarboxynaphtalenic acid. Other organic compounds that can be used as pesonal care ingredients include those described in U.S. Pat. No. 6,509,008 to Candau, of which the text of column 3, line 54 through column 12, line 37 are hereby incorporated by reference. Many of the personal care ingredients can be attached to, for example, a nitrogen containing species and can be quaternized and attached to the POSS molecule, directly or through some additional bridging group using, for example, epichlorhydrine as discussed previously.

The amount of personal care ingredients will vary widely with the type of personal care product, the type and amount of the other components used in the personal care product, the amount and type of EPOSS used and other factors well-known to formulators. However, in general, the amount of personal care ingredients useful in accordance with the present invention is the balance of the personal care product (everything other than EPOSS). The amount of personal care ingredients accordance with the present invention generally will be no more than 99.995%, preferably about 99.99%, more preferably 99.95%, and even more preferably about 99.9%. Furthermore, the amount of personal ingredients will generally be at least about 10%, preferably at least about 60%, more preferably at least about 80%, and even more preferably at least about 90%.

The functions of EPOSS molecules in personal care products can vary widely with the type of EPOSS used, the product, the amounts used, etc. In hair, for example, EPOSS compounds may be used as adhesive spheres that are useful in hair styling. They may be beneficial cosmetically acceptable carriers for depositing functional materials on to hair fiber. As adhesive materials, EPOSS molecules could also be used to enhance the long-wear properties of hair coloration. They would also be applicable to non-transfer properties of foundation and lipstick, and to nail polish and enamel. EPOSS could be used as active delivery vehicles for skin applications. EPOSS could be chemically bonded to skincare actives.

EPOSS molecules can be used to compatibilize various ingredients in a personal care product. For example, the cage like structure of a EPOSS can be formed around a dye, pigment molecule or particle, or pearlescent powder material. Molecules can exist partially within and partially outside of an EPOSS cage which may also assist in compatibilizing materials. The EPOSS could be derivatized such that its R groups are relatively more soluble in a solvent or in the remaining formulation than the material within, mixed with, or attached to the cage. This phenomenon has been observed with other cage molecules such as cyclodextrins. Alternatively, otherwise insoluble or incompatible ingredients could be bound to one or more corners of the EPOSS molecule, the resulting molecule being comparatively more compatible than the initially insoluble ingredient alone. Indeed, the derivative groups that are attached to the corners of the EPOSS molecule can be modified to tailor the ability of the molecule to solubilize or compatibilize other ingredients. By attaching hydrophilic or hydrophobic groups, changes can be made in the overall hydrophilic/lipophilic balance. In fact, the mere presence of certain EPOSS molecules, even those not bound to or containing an insoluble material in a system could provide advantages in terms of solubility or compatibility much like a surfactant does.

By derivatizing the basic EPOSS molecule, its function can be modified in any number of ways. For example, one or more quaternary nitrogens can be attached to an EPOSS molecule, or polymer backbone containing EPOSS molecules, to provide a positive overall charge to the resulting molecule. Such derivatives can provide all the known advantages of quaternary compounds such as, for example, great affinity for the keratins. EPOSS moieties, particularly as pendent groups in polymers, due to their non bonded interactions, such as by van der Waals, steric, repulsive forces, can align parallel on the keratinous surfaces and create, enhance or protect the film formed by the polymer. Compounds such as these may have added advantages including the ability to be retained in the hair, even after rinsing. EPOSS molecules assist in repairing the structural integrity of certain physical structures such as hair or may be able to permeate into cracks in the hair structure. EPOSS may be able to attach to various sides of a rift in the hair structure, helping to glue the structure together. EPOSS molecules may attach to the sulfide group of cystine amino acids found in hair retarding the reformation of disulfide bonds characteristic of curly hair. The exact opposite phenomenon may be encouraged. EPOSS molecules can be provided with multiple sulfur containing substituents increasing the likelihood that various cystine molecules will bond, albeit through a bridge of a EPOSS molecule.

In one embodiment in accordance with the present invention, EPOSS molecules can be used as structural components in film formers and in formulations such as, for example, nail polish. Depending on the type of EPOSS molecule selected and its potential reactivity, EPOSS molecules can add structure to a nail polish by providing more rigid structural components. Polymers made using EPOSS may also be used for increased strength and durability. Moreover, EPOSS molecules may act as an interface between nail polish and the nail, depending upon how they are substituted. EPOSS molecules can be engineered to have better adhesion to a nail than the enamel or polish itself, such as by being substituted with epoxide or thiol group which can covalently bond forming a covalently bond film. Similarly, EPOSS molecules can be used in conjunction with film formers, both because they can exhibit film forming properties, but also because they can add strength to the resulting film. The use of certain EPOSS containing certain R groups, such as epoxide or thiol, may also react with the surface of nails and form a covalently bonded film.

Cosmetic products in accordance with the present invention can be made using a “cosmetically acceptable medium,” which is often made up of one or more personal care ingredients. Generally, these may be composed of a fatty phase, optionally organic solvents and optionally water or other solvents in amounts such that they do not interfere with the EPOSS.

The fatty phase may be made of generally fatty substances that are liquid at room temperature (generally 25° C.) and/or of fatty substances that are solid at room temperature, such as waxes, gums and pasty fatty substances, and mixtures thereof.

As fatty substances that are liquid at room temperature, often referred to as oils, which may be used in the invention, mention may be made of silicone oils, hydrocarbon-based oils, of mineral, animal, plant or synthetic origin, alone or as a mixture provided that they form a homogeneous and stable mixture and provided that they are compatible with the intended use.

The cosmetically acceptable medium preferably contains volatile and/or non-volatile silicone oils.

Non-volatile silicone oils which may be mentioned are polydimethylsiloxanes (PDMSs), that are optionally phenylated, such as phenyltrimethicones, phenyltrimethyl-siloxydiphenylsiloxanes, diphenylmethyldimethyltrisiloxanes, diphenyldimethicones, phenyldimethicones and polymethylphenylsiloxanes, optionally substituted with aliphatic and/or aromatic groups, or optionally fluorinated; polysiloxanes modified with fatty acids, fatty alcohols or polyoxyalkylenes, fluorosilicones and perfluorosilicone oils, and mixtures thereof.

Among the non-volatile silicone oils that are useful, mention may be made of polydimethylsiloxanes, polymethylphenylsiloxanes, silicones comprising polyoxyalkylene blocks or grafts, in particular polyoxyethylene or copoly(oxyethylene/oxypropylene) blocks or grafts, such as dimethicone copolyols, silicones bearing both hydrophobic hydrocarbon-based groups (for example C₂-C₃₀ alkyl groups) and polyoxyethylenated or copoly(oxyethylenated/oxypropylenated) blocks or grafts, such as alkyldimethicone copolyols, silicones bearing fluoro or perfluoro groups such as perfluoroalkyl polydimethylsiloxanes and perfluoroalkyl polymethylphenylsiloxanes, and mixtures thereof.

One or more oils that are volatile at room temperature may also be used. These volatile oils make it easier to apply the composition to the skin, the lips and superficial body growths.

The term “volatile oil” means an oil which is capable of evaporating at the temperature of the skin or the lips, and which has a non-zero vapour pressure at room temperature and under atmospheric pressure, ranging in particular from 0.13 to 4.0×10⁴ Pa (10⁻³ to 300 mm Hg) and better still greater than 40 Pa (0.3 mm Hg).

These oils may be silicone oils optionally comprising alkyl or alkoxy groups at the end of or pendent on a silicone chain.

As volatile silicone oils which may be used in the invention, mention may be made of linear or cyclic silicones with a viscosity at room temperature and under atmospheric pressure of less than 8 mm²/s (8 cSt) and in particular comprising from 2 to 7 silicon atoms. Mention may be made in particular of octamethylcyclotetrasiloxane, decamethylcyclopentasiloxane, hexadecamethylcyclohexasiloxane, heptamethylhexyltrisiloxane and heptamethyloctyltrisiloxane, and mixtures thereof.

Preferably, at least one volatile silicone oil chosen especially from octamethylcyclotetrasiloxane, decamethylcyclopentasiloxane, hexadecamethylcyclohexasiloxane, heptamethylhexyltrisiloxane, heptamethyloctyltrisiloxane, octamethyltrisiloxaneand decamethyltetrasiloxane, and mixtures thereof, may be used.

The cosmetically acceptable medium may also contain one or more oils of non-silicone nature such as, for example, hydrocarbon-based oils.

The term “hydrocarbon-based oil” means an oil predominantly containing carbon and hydrogen atoms, and in particular alkyl or alkenyl chains such as alkanes or alkenes, as well as an oil not only containing hydrogen and carbon atoms, but also oxygen atoms, in the form of an ether, ester, alcohol or carboxylic acid function.

Mention may also be made of hydrocarbon-based oils such as liquid paraffin or liquid petroleum jelly, mink oil, turtle oil, soyabean oil, perhydrosqualene, sweet almond oil, beauty-leaf oil, palm oil, grape pip oil, sesame oil, corn oil, parleam oil, arara oil, rapeseed oil, sunflower oil, cottonseed oil, apricot oil, castor oil, avocado oil, jojoba oil, olive oil or cereal germ oil; esters of linoleic acid, of oleic acid, of lauric acid or of stearic acid; fatty esters, such as isopropyl myristate, isopropyl palmitate, butyl stearate, hexyl laurate, diisopropyl adipate, isononyl isononanoate, 2-ethylhexyl palmitate, 2-hexyldecyl laurate, 2-octyldecyl palmitate, 2-octyldodecyl myristate or lactate, bis(2-ethylhexyl) succinate, diisostearyl malate and glyceryl or diglyceryl triisostearate; higher fatty alcohols containing at least 12 carbon atoms, such as stearyl alcohol, oleyl alcohol, linoleyl alcohol, linolenyl alcohol, isostearyl alcohol or octyldodecanol.

For the purposes of the present invention, a wax is a lipophilic compound which is solid at room temperature (about 25° C.), which undergoes a reversible solid/liquid change of state, which has a melting point above about 40° C. which may be up to 200° C., and which has an anisotropic crystal organization in the solid state. In general, the size of the wax crystals is such that the crystals scatter and/or diffuse light, giving the composition comprising them a more or less opaque cloudy appearance. By bringing the wax to its melting point, it is possible to make it miscible with oils and to form a microscopically homogeneous mixture, but on returning the temperature of the mixture to room temperature, a recrystallization of the wax in the oils of the mixture is obtained, which may be detected microscopically and macroscopically (opalescence).

As examples of waxes which may be used according to the invention, mention may be made of waxes of animal origin such as beeswax, spermaceti, lanolin wax and lanolin derivatives; plant waxes such as carnauba wax, candelilla wax, ouricury wax, Japan wax, cocoa butter, cork fibre wax of sugar cane wax; mineral waxes, for example paraffin wax, petroleum jelly wax, lignite wax, microcrystalline waxes or ozokerites; synthetic waxes including polyethylene wax, polytetrafluoroethylene wax and the waxes obtained by Fisher-Tropsch synthesis, or alternatively silicone waxes, hydrogenated oils that are solid at 25° C., such as hydrogenated castor oil, hydrogenated jojoba oil, hydrogenated palm oil, hydrogenated tallow and hydrogenated coconut oil, and fatty esters that are solid at 25° C., such as the C₂₀-C₄₀ alkyl stearate sold under the trade name Kester Wax K82H by the company Koster Keunen.

The gums are generally high molecular weight polydimethylsiloxanes (PDMSs) and the pasty substances are generally hydrocarbon-based compounds, for instance lanolins and derivatives thereof, or PDMSs.

The cosmetic composition and personal care products according to the invention may include one or more cosmetically acceptable (acceptable tolerance, toxicology and feel) organic solvents. These organic solvents may be chosen from hydrophilic organic solvents, lipophilic organic solvents and amphiphilic solvents, and mixtures thereof.

Among the hydrophilic organic solvents which may be mentioned, for example, are linear or branched lower monoalcohols containing from 1 to 8 carbon atoms, for instance ethanol, propanol, butanol, isopropanol, isobutanol; acetone; polyethylene glycols containing from 6 to 80 ethylenoxy units; polyols such as propylene glycol, butylene glycol, glycerol or sorbitol; esters such as ethylacetate and methylacetate, mono- or dialkyl isosorbides in which the alkyl groups contain from 1 to 5 carbon atoms, for instance, dimethyl isosorbide; for instance, diethylene glycol monomethyl ether or monoethyl ether and propylene glycol ethers, such as dipropylene glycol methyl ether.

Other organic solvents which may be mentioned include polyols such as polypropylene glycol (PPG) derivatives, for instance polypropylene glycol esters of fatty acids, and PPG ethers of fatty alcohols, for example PPG-36 oleate and PPG-23 oleyl ether.

Lipophilic organic solvents which may be mentioned, for example, include hydrocarbons such as hexane, heptane and octane; fatty esters such as diisopropyl adipate and dioctyl adipate; alkyl benzoates; dioctyl malate.

A person skilled in the art will take care to select the optional additives and the amount thereof such that they do not harm the properties of the compositions.

Fillers may be used and these fillers may be mineral or organic, and lamellar or spherical. Mention may be made of talc, mica, silica, kaolin, Nylon® (Orgasol® from Atochem) powder, polyalanine powder and polyethylene powder, Teflon®, lauroyllysine, starch, boron nitride, hollow microspheres such as Expancel® (Nobel Industry), Polytrap® (Dow Corning) and silicone resin microbeads (Tospearls® from Toshiba, for example), precipitated calcium carbonate, magnesium carbonate and magnesium hydrocarbonate, hydroxyapatite, hollow silica microspheres (silica beads from Maprecos), glass or ceramic microcapsules, metal soaps derived from organic carboxylic acids containing from 8 to 22 carbon atoms and preferably from 12 to 18 carbon atoms, for example zinc, magnesium or lithium stearate, zinc laurate or magnesium myristate.

Pigments useful in the cosmetic compositions or personal care products on the invention may be white or coloured, and mineral and/or organic. Among the mineral pigments which may be mentioned are titanium dioxide, optionally surface-treated, zirconium oxide or cerium oxide, and iron oxide or chromium oxide, manganese violet, ultramarine blue, chromium hydrate and ferric blue. Among the organic pigments which may be mentioned are carbon black, pigments of D&C type, and lakes based on cochineal carmine or on barium, strontium, calcium or aluminum.

Nacreous pigments may be used and may be chosen from white nacreous pigments such as mica coated with titanium oxide or with bismuth oxychloride, coloured nacreous pigments such as titanium mica with iron oxides, titanium mica with, in particular, ferric blue or chromium oxide, titanium mica with an organic pigment of the abovementioned type and nacreous pigments based on bismuth oxychloride.

Liposoluble colorants may be used and include, for example, Sudan Red, DC Red 17, DC Green 6, β-carotene, DC Yellow 11 or DC Violet 2. They may represent from 0.01% to 20% of the weight of the composition and better still from 0.1% to 6%.

In one embodiment of an EPOSS containing nail varnish of the present invention, cellulose acetopropionates can be present in an amount ranging from 10% to 80% by weight, relative to the total weight of cellulose ester and nitrocellulose present in the composition. In another embodiment of the invention, cellulose acetopropionates can be present in the nail varnish composition in an amount ranging from 12.5% to 75% by weight, relative to the total weight of cellulose ester and nitrocellulose present in the composition.

Nitrocellulose can be present in the composition in an amount ranging from 1.5 to 35% by weight relative to the total weight of the composition. In another embodiment, the nitrocellulose can be present in the composition in an amount ranging from 8% to 20% by weight, relative to the total weight of the composition. Nitrocellulose and cellulose esters useful in accordance with the present invention are identified in U.S. Pat. No. 6,333,025 to Ramin, the text of which is hereby incorporated by reference.

According to another embodiment of the invention, the composition comprises at least one film-forming polymer in addition to nitrocellulose and cellulose ester, at least to improve the cosmetic and physicochemical properties of nail varnish film.

In yet another embodiment of the invention, the additional film-forming polymer, which may include EPOSS, can be present in the nail varnish composition in an amount up to 50% by weight, and is preferably present in an amount less than 40% by weight, relative to the total weight of nitrocellulose and cellulose ester. In still another embodiment of the invention, the amount of additional film forming polymer ranges from 1% to 15% by weight relative to the total weight of nitrocellulose and cellulose ester.

The solvent medium for the nail varnish composition can comprise at least one organic solvent.

Examples of organic solvents which can be used in the invention include:

ketones which are liquid at room temperature, such as methyl ethyl ketone, methyl isobutyl ketone, diisobutyl ketone, isophorone, cyclohexanone and acetone;

alcohols which are liquid at room temperature, such as ethanol, isopropanol, diacetone alcohol, 2-butoxyethanol and cyclohexanol;

glycols which are liquid at room temperature, such as ethylene glycol, propylene glycol, pentylene glycol and glycerol;

propylene glycol ethers which are liquid at room temperature, such as propylene glycol monomethyl ether, propylene glycol monomethyl ether acetate and dipropylene glycol mono-n-butyl ether;

short-chain esters (containing from 3 to 8 carbon atoms in total), such as ethyl acetate, methyl acetate, propyl acetate, n-butyl acetate and isopentyl acetate;

ethers which are liquid at room temperature, such as diethyl ether, dimethyl ether and dichlorodiethyl ether;

alkanes which are liquid at room temperature, such as decane, heptane, dodecane and cyclohexane;

aromatic cyclic compounds which are liquid at room temperature, such as toluene and xylene;

aldehydes which are liquid at room temperature, such as benzaldehyde and acetaldehyde, and mixtures thereof.

Compositions according to the invention can further comprise water in an amount ranging up to 10% by weight, relative to the total weight of the composition.

Compositions according to the invention may also comprise at least one plasticizer. Examples of plasticizers suitable for use in the present invention, alone or as a mixture, include:

glycols and derivatives thereof such as diethylene glycol ethyl ether, diethylene glycol methyl ether, diethylene glycol butyl ether, diethylene glycol hexyl ether, ethylene glycol ethyl ether, ethylene glycol butyl ether and ethylene glycol hexyl ether;

glycerol esters;

propylene glycol derivatives including propylene glycol phenyl ether, propylene glycol diacetate, dipropylene glycol butyl ether, tripropylene glycol butyl ether, propylene glycol methyl ether, dipropylene glycol ethyl ether, tripropylene glycol methyl ether, diethylene glycol methyl ether and propylene glycol butyl ether;

acid esters, including carboxylic acid esters, such as citrates, phthalates, adipates, carbonates, tartrates, phosphates and sebacates;

oxyethylenated derivatives, including oxyethylenated oils, for example, plant oils such as castor oil; and mixtures thereof.

The amount of plasticizer can be chosen by a person skilled in the art on the basis of his or her general knowledge, so as to obtain a composition which has cosmetically acceptable properties. The amount of plasticizer present in the composition can range, for example, from 0.5% to 20% by weight relative to the total weight of the composition. In an embodiment of the invention, the amount of plasticizer ranges from 2% to 10% by weight relative to the total weight of the composition.

The dyestuff present in the composition can be chosen from pulverulent compounds and dyes which are soluble in the solvent medium of the composition, in an amount which is sufficient to dye the varnish in a color in the visible wavelength range, i.e., between 400 and 800 nm, and, for example, in a content ranging from 0.001% to 10% by weight relative to the total weight of the composition. The pulverulent compounds can be chosen from the pigments, nacres and glitter flakes usually used in nail varnishes.

The pigments can be white or colored, and inorganic or organic. Examples of inorganic pigments include titanium dioxide, which has optionally been surface-treated, zirconium oxide and cerium oxide, as well as iron oxide and chromium oxide, manganese violet, ultramarine blue, chromium hydrate and ferric blue, and metallic pigments such as aluminum and bronze. Examples of organic pigments include carbon black, pigments of D&C type and lakes based on cochineal carmine, barium, strontium, calcium, aluminum, and guanine.

The nacreous pigments can be chosen from white nacreous pigments such as mica coated with titanium or with bismuth oxychloride, colored nacreous pigments such as titanium mica with, for example, iron oxides, ferric blue, chromium oxide, or with an organic pigment of the above-mentioned type, as well as nacreous pigments based on bismuth oxychloride.

The glitter flakes can be chosen from those made of acrylic, of polyester and of polyethylene terephthalate, and of aluminum.

The dyes are, for example, Sudan red, DC Red 17, DC Green 6, α-carotene, soybean oil, Sudan brown, DC Yellow 11, DC Violet 2, DC Orange 5 and cannelloni yellow.

EPOSS containing nail varnish compositions of the invention can also optionally comprise an optical brightener, for example to reduce the yellowing effect of the nitrocellulose and consequently of the composition, this brightener not being considered, for the purposes of the invention, as a dyestuff. One brightener which can be used is the monosodium salt of alizurol purple; this brightener can be present in the inventive compositions in an amount ranging from 10⁻⁷% to 10⁻⁴% by weight relative to the total weight of the composition.

EPOSS containing nail varnish compositions according to the invention can also comprise any additive known to those skilled in the art capable of being incorporated into dye compositions. Such additives include thickeners, spreading agents, wetting agents, dispersing agents, antifoaming agents, preserving agents, UV screening agents, active agents, surfactants, moisturizers, fragrances, neutralizers, stabilizers, and antioxidants. Needless to say, a person skilled in the art will take care to select this or these optional additional compounds, and/or the amount thereof, such that the advantageous properties of the composition for the use according to the invention are not, or are not substantially, adversely affected by the addition envisaged. In an embodiment of the invention, the composition is free or essentially free of silicone drying agents. See also U.S. Pat. No. 6,372,201 to Leuridan et al., the text of which is also incorporated by reference. The amount of EPOSS used in any nail polish may vary. Preferably, however, the amount ranges from about 0.005 to about 40% w/w. More preferably about 0.05 to about 25% (w/w). Even more preferably about 0.1 to about 20% (w/w). These same amounts may be used in other nail products as polishes and enamels as well and indeed most personal care products.

Indeed, EPOSS may be added to any of the nail varnish compositions of U.S. Pat. No. 6,333,025, and any other known nail polish, varnish or enamel. Nail enamels that may be used in accordance with the present invention include those disclosed in U.S. Pat. No. 6,555,096.

Nail enamel compositions typically contain, in an organic solvent or mixture of solvents, film-forming ingredients, plasticizing ingredients, and colorants. Generally, the composition also contains a thixotropic agent, which may act to thicken the composition in order to allow better spreading on the nail. The thixotropic agent may also act to suspend the colorant.

The classic thixotropic agent used in the prior art is a bentonite clay. Aromatic organic solvents in particular may cause these clays to swell, thus providing a gel with good thixotropic properties, i.e., rendering the composition capable of passing from a gelled state to a liquid state simply by stirring and capable of going from liquid to gel after standing. A composition containing such a gel thus may exhibit relatively good dispersion stability without sedimentation or separation over a long Period. Further, such compositions may not require the vigorous shaking that other compositions often require after extended periods of storage. Urea-modified thixotropic agents made from modified urethanes are disclosed in U.S. Pat. No. 6,555,096 to Carrion, the text of which is hereby incorporated by reference.

EPOSS containing nail enamel compositions preferably include a cosmetically acceptable solvent system comprising diacetone alcohol and at least one additional solvent chosen from C₁-C₆ alkyl acetates and C₁-C₆ alkyl alcohols; at least one film-forming substance; and at least one thixotropic agent. The nail enamel composition of the invention may also contain a plasticizer and optionally a coloring agent as described for varnishes.

The nail enamel composition of the invention may contain at least one additional thixotropic agent, used in conjunction with the at least one urea-modified thixotropic agent. When such an additional thixotropic agent is present, the composition may comprise from about 0.10 to about 0.30% of the at least one urea-modified thixotropic agent relative to the weight of the composition and up to about 1.0% of the additional thixotropic agent. The additional thixotropic agent(s) may be chosen from conventional silica and bentonite clay agents. It is also possible to use these additional thixotropic agents instead of urea-modified agents as desired.

Film forming substances useful in the present invention include, but are not limited to, conventional film-forming agents such as nitrocellulose, other cellulose derivatives, such as cellulose acetate, cellulose acetate butyrate, and ethyl cellulose; polyesters; resins, such as polyurethane resins, alkyd resins, and polyvinyl resins such as polyvinyl acetate, polyvinyl chloride, polyvinylbutyrate; (meth)acrylic and vinyl copolymers such as styrene/butadiene copolymers, acrylate/vinyl acetate copolymers, acrylonitrile/butadiene copolymers, and ethylene/vinyl acetate copolymers.

In one embodiment, the primary film-forming agent may be nitrocellulose, which is known to give hardness and resistance to abrasion. If a second film-forming substance is present, this second film-forming substance may, for example, be chosen from cellulose acetate butyrate and acrylates. In one embodiment, the film-forming substance may be present in an amount of from about 5% to about 20% by weight relative to the weight of the composition, for example, from about 10% to about 14%. Suitable modifiers for the primary film-forming agent include arylsulfonamide resins such as arylsulfonamide formaldehyde or epoxy resins.

The presently claimed nail enamel composition including EPOSS also may contain at least one plasticizer. Plasticizers useful in the presently claimed nail enamel composition include plasticizers commonly employed in nail varnish compositions. These plasticizers encompass, but are not limited to, dibutyl phthalate, dioctyl phthalate, tricresyl phthalate, butyl phthalate, dibutoxy ethyl phthalate, diamylphthalate, tosyl amide, N-ethyl-tosyl amide, sucrose acetate isobutyrate, camphor, castor oil, citrate esters, glyceryl diesters, glyceryl triesters, tributyl phosphate, tri-phenyl phosphate, butyl glycolate, benzyl benzoate, butyl acetyl ricinoleate, butyl stearate, and dibutyl tartrate. In one embodiment, a plasticizer used in the present invention may be the mixture of acetyl tributyl citrate and N-ethyl tosyl amide. The plasticizer may, for example, be present in an amount of from about 3% to about 12% by weight relative to the weight of the composition.

The cosmetically acceptable solvent system of the nail enamel of the present invention comprises diacetone alcohol and at least one additional solvent chosen from C₁-C₆ alkyl acetates and C₁-C₆ alkyl alcohols. In one embodiment, the C₁-C₆ alkyl acetates are chosen from ethyl acetate, propyl acetate, and butyl acetate. In another embodiment, the C₁-C₆ alkyl alcohols are chosen from ethanol, isopropanol, and butanol. Other cosmetically acceptable organic solvents which can be used in conjunction with the inventive solvent system include, but are not limited to, toluene; xylene; ketones such as acetone or methyl ethyl ketone; glycol ethers; alkanes such as hexane or heptane; -methylpyrrolidone; and alkyl lactates. The solvent system of the invention, including any additional solvents, may be present, in one embodiment, in an amount of from about 40% to about 80% by weight relative to the weight of the composition, and, in another embodiment, from about 65% to about 78%.

The nail enamel composition of the invention may also contain at least one coloring agent. Conventional coloring agents can be used, and examples include inorganic pigments such as titanium dioxide, iron oxides, titanated mica, iron oxide coated mica, ultramarine, chromium oxide, chromium hydroxide, manganese violet, bismuth oxychloride, guanine, and aluminum; pearlescent materials; and organic coloring agents such as ferric ammonium ferrocyanide, and D&C Red Nos. 6, 7, 34, Blue No. 1, Violet No. 2, and Yellow No. 5.

The inorganic pigments may be surface-treated as is customary to prevent migration or striation. Silicones and polyethylenes are most often used as the coatings for inorganic pigments and thus may be used according to the present invention. Colorant materials may also include chips or powder of mica or diamonds in the nail composition. Also useful are specialty materials giving rise to two-tone color effects such as liquid crystal silicones or multi-lamellar metallic particulates, which generally can be mixed with pigments or dyes to obtain a broader spectrum of brilliant color and increased luminous reflectance. Such materials are described in, e.g., U.S. Pat. Nos. 3,438,796; 4,410,570; 4,434,010; 4,838,648; 4,930,866; 5,171,363; 5,364,467; 5,569,535; 5,607,904; 5,624,486; 5,658,976; 5,688,494; 5,766,335; N. Hatberle et al., “Right and Left Circular Polarizing Colorfilters made from Crosslinkable Cholesteric LC-Silicones,” Conference Record of the 1991 International Display Research Conference (IEEE), pp. 57-59; R. Maurer et al., “Polarizing Color Filters made from Cholesteric LC-Silicones,” SID 90 Digest (1990), pp. 110-113; H.-J. Eberle et al., “Inverse Angle Dependence of the Reflection Colours of Cholesteric Polymeric Liquid Crystals Mixed with Pigments,” Liquid Crystals, 5(3), (1989), pp. 907-916; J. Pinsl et al., “Liquid Crystalline Polysiloxanes for Optical Once-Write Storage,”¹ J. Molec. Electr., Vol. 3 (1987), pp. 9-13; and D. Makow, “Reflection and Transmission of Polymer Liquid-Crystal Coatings and their Application to Decorative Arts and Stained Glass,” Color Res. Applic. Vol. 11, No. 3, (1986), pp. 205-208, all of which are incorporated herein by reference in their entirety.

In one embodiment, the coloring agent may be present in the nail enamel composition in an amount up to about 5% by weight relative to the total weight of the composition. In another embodiment, the coloring agent is present in an amount of from 2% to 3% by weight.

The composition according to the invention may also include additives recognized by a person skilled in the art as being capable of incorporation into such a composition. For example, the composition may include at least one cosmetically active compound, which may be selected from vitamins, minerals, moisturizers, flavoring compounds, fragrances, masking agents, hardening agents such as silica and formaldehyde/glyoxal, UV absorbers, and fibers such as nylon and aramide fibers. Any art-recognized UV absorber can be used, both organic and inorganic. In one embodiment, inorganic UV absorbers include titanium dioxide and zinc oxide, both of which may be used in nanoparticulate form. In another embodiment organic UV absorbers include octocrylene, octylmethoxy cinnamate, and benzophenone.

For hair, EPOSS containing compositions in accordance with the invention are more particularly shampoos, rinse-out or leave-in compositions to be applied before or after a shampooing, conditioners, dyeing, bleaching, permanent-waving or hair-straightening operation, or dyeing, bleaching, permanent-waving or straightening compositions for the hair.

The compositions can also be hairsetting and styling lotions, blow-drying lotions, mousses or fixing lacquers and/or styling compositions.

The ability to create molecules based on an EPOSS structure that is both hydrophilic and hydrophobic offers advantages in the creation of compatibilizers, emulsifiers and viscosity modifiers. EPOSS molecules can be reacted with one or more hydrophilic groups of various propensity for water, thereby adjusting the overall hydrophobic, lipophilic balance of the resulting material. The balance may be further tailored by adding additional groups that render the EPOSS molecule more hydrophobic as well. Having many reactive sites on which to place substituents of varying polarity and degrees of hydrophobicity and hydrophilicity provides a tremendous amount of flexibility in designing emulsifiers and compatibilizers for even the most challenging systems.

The present invention also relates to a method of treating hair. This includes the step of applying a composition defined above to the hair, distributing it homogeneously, and after leaving it on the hair for a suitable period, optionally rinsing and then drying the hair thus treated. Those of skill in the art will appreciate how to produce hair care products including EPOSS.

Another particularly preferred group of EPOSS molecules useful in accordance with the present invention are substituted with groups which can provide protection for hair, and in particular, skin, from electromagnetic radiation such as UV-A, UV-B and UV-C rays emanating from the sun. These EPOSS molecules can be used in both skin and hair care products. As mentioned above, such groups may be attached directly to one or more Si atoms or may be bound thereto through a bridging molecule. Because EPOSS molecules can be derivatized with a number of such UV protective groups, for example, a higher density of UV protection may be obtained. Furthermore, UV protecting and conditioning groups may both be attached to the same EPOSS nucleus, providing a composition useful for both purposes. Indeed, if a plurality of Si atoms in each EPOSS molecule with derivatized with conditioning quats and UV protecting groups, the result could be a UV protecting moiety with good conditioning and high substantivity when applied to the skin or hair. Such multifunctional EPOSS molecules may provide these advantages as well as the advantages that inure from the relatively higher density of UV absorbing groups and the ability to eliminate one or more ingredients typically found in personal care products of these desired functions as a single molecule can provide multiple functions. This provides much greater flexibility in terms of formulating the final product. In this instance, the amount of EPOSS molecule used will depend upon the desired degree of the predetermined personal care property which could be conditioning and/or UV protection.

In another embodiment, the present invention relates to a method of treating skin. This includes the step of applying a composition defined above to skin, distributing it homogeneously to treat the skin.

There are a number of specific formulations that may be useful when produced including the appropriate EPOSS. Examples of these formulations include, without limitation, a mixture of appropriate POSS materials, phenyl trimethicone (A) and laurylmethicone copolyol (B). These can be formulated into LAN serums to yield clear, dilute solutions. The nonionic surfactants can be PPG-5-Ceteth-20, Oleth-10, and also decyl glucoside. The amphoteric surfactant can be disodium cocoamphodipropionate (MIRANOL C2M-SF Conc.).

| Ingredient | Percentage A | Percentage B |
|---|---|---|
| EPOSS ® material | 0.05-5.00% | 1.00-10.00% |
| Lecithin | 4.00% | 4.00% |
| PPG-5-Ceteth-20 (nonionic | 14.00% | — |
| surfactant) | | |
| Oleth-10 (nonionic | — | 15.00% |
| surfactant) | | |
| Decyl glucoside | 15.00% | 10.00% |
| Disodium | 19.00% | 1.00% |
| cocoamphodipropionate | | |
| Water PH adjusted to 6.0-6.5 | q.s. to 100% | q.s. to 100% |
| with phosphoric acid | | |

The resulting EPOSS containing carriers could then be incorporated into styling gels, hair-shine gels, shampoos, conditioners, and other formulations. All ingredient amounts are shown in weight percentage.

A styling gel can be formulated using another formulation provided below where all ingredient amounts are shown in weight percentage.

| Ingredient | Percentage |
|---|---|
| Lecithin | 4.00% |
| Disodium cocoamphodipropionate | 19.00% |
| (amphoteric surfactant) | |
| PPG-5-Ceteth-20 (nonionic surfactant) | 14.00% |
| Oleth-10 (nonionic surfactant) | 9.00% |
| *EPOSS | 1.00-5.00% |
| Guar hydroxypropyltrimonium chloride | 0.50% |
| (thickening agent) | |
| Water | q.s. to 100% |

*The appropriate EPOSS will generally be selected from EPOSS ® Molecular Silicas ™; EPOSS ® Functionalized Monomers; EPOSS ® Silanols; EPOSS ® Polymers and Resins.

Similarly, a hair shine gel can be formulated. The polymer used in the gels can be VISCOPHOBE DB-1000, an acrylic acid latex from Rhodia. All ingredient amounts are shown in weight percentage.

| Ingredient | Percentage |
|---|---|
| ALCOLEC F100 | 1.00% |
| MIRANOL C2M-SF CONC | 10.00% |
| ARLASOLVE 200 | 15.00% |
| *EPOSS | 1.00-5.00% |
| VISCOPHOBE DB-1000 | 12.00% |
| Water | q.s. to 100% |

*The appropriate EPOSS will generally be selected from EPOSS ® Molecular Silicas ™; EPOSS ® Functionalized Monomers; EPOSS ® Silanols; EPOSS ® Polymers and Resins.

In another embodiment, selected hair swatches can be wrapped around permanent waving rods, saturated with the following solutions:

| Ingredient | Percentage |
|---|---|
| Thioglycolic acid | 1.50% |
| ALCOLEC F100 (lecithin) | 0.06% |
| MIRANOL C2M-SF CONC. (amphoteric) | 0.18% |
| ARLASOLVE 200 (nonionic) | 0.25% |
| AMPHOMER LV-71 | 1.00% |
| Aminomethylpropanol (AMP) | 0.13% |
| *EPOSS | 1.00-5.00% |
| Water | 93.38˜97.38% |
| Ammonium Hydroxide | q.s. to pH 9.2 |

*The appropriate EPOSS will generally be selected from EPOSS ® Molecular Silicas ™; EPOSS ® Functionalized Monomers; EPOSS ® Silanols; EPOSS ® Polymers and Resins.

After about 30 minutes at room temperature, the hair swatches (still on the rods) can be thoroughly rinsed with water and blotted dry. They can be treated with a 2% hydrogen peroxide solution for 5 minutes at room temperature, rinsed thoroughly with water and blotted dry. The hair swatches can be removed from the rods. Again, all amounts recited are in weight percentage. Lower percentages of MIRANOL and VISCOPHOBE are also contemplated.

A clear shampoo for color treated hair using EPOSS can be formulated. It can contain a EPOSS material carrier. All ingredient amounts are shown in weight percentage.

| Ingredient | Percentage |
|---|---|
| Lecithin | 4.00% |
| Disodium cocoamphodipropionate | 19.00% |
| (amphoteric surfactant) | |
| PPG-5-Ceteth-20 (nonionic surfactant) | 14.00% |
| Oleth-10 (nonionic surfactant) | 9.00% |
| Methyl paraben | 0.20% |
| Ethyl paraben | 0.10% |
| Disodium EDTA | 0.10% |
| Pheonoxyethanol | 0.50% |
| Phosphoric acid 85% (pH adjuster) | 1.40% |
| *EPOSS | 1.00-5.00% |
| Octyl salicylate (sunscreen) | 0.50% |
| Water | q.s. to 100% |

*The appropriate EPOSS will generally be selected from EPOSS ® Molecular Silicas ™; EPOSS ® Functionalized Monomers; EPOSS ® Silanols; EPOSS ® Polymers and Resins.

0.100%-1.00% of the above carrier can be used in a shampoo base of:

| | |
|---|---|
| Sodium laureth sulfate (anionic | 25.000% |
| surfactant) | |
| Polyquaternium 10 (polymer) | 0.100% |
| PPG-5-Ceteth-10-phosphate emollient | 0.500% |
| Disodium cocoamphodipropionate and | 13.000% |
| cocamidopropyl betaine | |
| (amphoteric surfactants) | |
| Octyl methoxy cinnamate (sunscreen) | 0.100% |
| Phosphoric acid (85%) | 0.800% |
| Hydrochloride lauryldimonium | 0.400% |
| hydroxypropyl hydrolyzed soy | |
| protein, and hydrolyzed soy protein | |
| with wheat amino acid | |
| Water | q.s. to 100% |

Similarly, EPOSS may be included in an emulsified cream treatment containing cationic conditioners, cationic polymers, sunscreens and Vitamin E, which can be formulated as follows:

| Ingredient | Percentage |
|---|---|
| Lecithin | 4.00% |
| Disodium cocoamphodipropionate | 19.00% |
| (amphoteric surfactant) | |
| PPG-5-Ceteth-20 (nonionic | 14.00% |
| surfactant) | |
| Oleth-10 (nonionic surfactant) | 9.00% |
| Methyl paraben | 0.20% |
| Ethyl paraben | 0.10% |
| Disodium EDTA | 0.10% |
| Pheonoxyethanol | 0.50% |
| Phosphoric acid 85% (pH adjuster) | 1.40% |
| *EPOSS | 1.00-5.00% |
| Water | q.s. to 100% |
| Vitamin E (tocopherol) | 1.00% |
| Octyl salicylate (sunscreen) | 1.00% |

*The appropriate EPOSS will generally be selected from EPOSS ® Molecular Silicas ™; EPOSS ® Functionalized Monomers; EPOSS ® Silanols; EPOSS ® Polymers and Resins.

0.100-1.00% of the above composition was in a conditioner base of:

| | |
|---|---|
| Glyceryl stearate and PEG-100 | 5.000% |
| stearate (nonionic emulsifier) | |
| Quaternium 27 | 4.000% |
| Hexadimethrine chloride and | 1.300% |
| hydroxyethyl cellulose | |
| (cationic and cellulosic polymers) | |
| Octyl methoxycinnamate (sunscreen) | 0.100% |
| Stearyl alcohol (emollient) | 5.000% |
| Octyldodecanol (emollient) | 2.000% |
| Sodium citrate | 0.150% |
| Water | q.s. to 100% |
| Fragrance | 0.500% |
| Preservatives | 0.900% |

In this context, the “appropriate EPOSS” is dictated by the many factors previously described including, without limitation, the predetermined personal care property, solubility, cost, reactivity with other ingredients and the like.

### EXAMPLES

### Example 1

### Sunscreen Cream

A sunscreen cream can be formulated. All ingredient amounts are shown in weight percentage.

| | |
|---|---|
| Glycerin | 20.00% |
| Camphor benzalkonium methosulfate | 6.60% |
| Isopropyl myristate | 3.00% |
| Cetylstearylic alcohol | 2.40% |
| 3-(4-methyl benzylidene)2-bornanone/ | 1.50% |
| 1-(4-isopropyl phenyl)3-phenyl 1,3- | |
| propanediol | |
| gylceryl stearate | 1.00% |
| PDMS | 1.00% |
| Cetylic alcohol | 1.00% |
| Cetearyl alcohol | 0.60% |
| Fragrance | 0.50% |
| POSS(octa(phenyl)octasilsesquioxane) | 0.50% |
| Castor oil | 0.30% |
| EDTA | 0.10% |
| Ditertiobutyl 4-hydroxytoluene | 0.05% |
| Water | 61.45% |

### Example 2

### Gel

A gel can be formulated. All ingredient amounts are shown in weight percentage.

| | |
|---|---|
| Glycerin | 35.00% |
| Polyacrylic acid | 0.75% |
| POSS(octa(phenyl)octasilsesquioxane) | 0.50% |
| Phenoxyethanol | 0.50% |
| Fragrance | 0.40% |
| PEG-40 hydrogenated castor oil | 0.30% |
| Water | 62.55% |

### Example 3

### Gel

Another type of gel can also be formulated. All ingredient amounts are shown in weight percentage.

| | |
|---|---|
| Glycerin | 35.00% |
| Polyacrylic acid | 0.75% |
| POSS(octa(phenyl)octasilsesquioxane) | 0.50% |
| Phenoxyethanol | 0.50% |
| PEG-40 hydrogenated castor oil | 0.30% |
| Benzophenone-4 | 0.02% |
| Fragrance | 0.10% |
| Water | 62.83% |

These formulations can be produced using conventional techniques known in the art.

Appropriate EPOSS molecules can be substituted for the POSS molecules identified in these examples. Applicant hereby incorporates by reference the entire text and claims of a concurrently filed U.S. patent application entitled “POSS CONTAINING COSMETICS AND PERSONAL CARE PRODUCTS” filed Mar. 12, 2004, naming Dr. Murat Quadir as inventor and filed under attorney docket number LOREAL 3.0-005 I.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A personal care product comprising: at least one EPOSS having a rigid three dimensional cage structure comprising nine or more Si atoms within its cage structure, said EPOSS being present in an amount effective to provide a desired degree of a predetermined personal care property; and
at least one personal care ingredient.

2. The personal care product of claim 1, formulated as a sunscreen composition, suntan product, antiperspirant, deodorant, cold cream, moisturizer, cleaner, shampoo, conditioner, dual shampoo/conditioner, rinse, cream rinse, cosmetic, hair coloring, hair dye, bleaching composition, styling product, cleansing cream, soap, perfume and cologne, powder, permanent waving product, relaxers, preshave, shaving cream, shaving product, after shaving product, bath product, self tanning product, bleaching product, and hair shine product

3. The personal care product as in any one of claims 1 and 2, formulated in the form of a liquid, solid, cream, ointment, solution, gel, mousse, stick, cream, spray, powder, emulsion or dispersion

4. The personal care product as in any one of claims 1 and 2, wherein said at least one EPOSS is present in an amount of at least about 0.005% by weight of said product.

5. The personal care product of claim 4, wherein said at least one EPOSS is present in an amount of at least about 0.01% by weight of said product.

6. The personal care product of claim 5, wherein said at least one EPOSS is present in an amount of at least about 0.05% by weight of said product.

7. The personal care product of claim 6, wherein said at least one EPOSS is present in an amount of at least about 0.10% by weight of said product.

8. The personal care product of claim 7, wherein said at least one EPOSS is present in an amount of about 40% or less by weight of said product.

9. The personal care product of claim 8, wherein said at least one EPOSS is present in an amount of about 20% or less by weight of said product.

10. The personal care product of claim 9, wherein said at least one EPOSS is present in an amount of about 10% or less by weight of said product.

11. The personal care products as in any one of claims 1 or 2, wherein said at least one personal care ingredient is an absorbent, alphahydroxy acid, betahydroxy acid, polyhydroxy acid, antiacne agent, antiperspirant, anticaking agent, antifoaming agent, antimicrobial agent, antioxidant, antidandruff agent, astringent, binder, bleaching agent, buffer, biological additive, bulking agent, carrier, chelating agent, coupling agent, compatibilizer, conditioner, colorant, cosmetic astringent, cosmetic biocide, denaturant, drug astringent, detergent, deodorant, dispersant, external analgesic, emulsifier, film former, foaming agent, fragrance and fragrance component, hair styling ingredient, hair holding ingredient (mousse, spray, etc.), hair conditioner, hair color, hair growth promoter, humectant, keratolytic, moisturizer, straightening agent, oxidizer, mineral and organic particle, plastic, polymer, permanent waving agent, opacifying agent, perfume, pH adjuster, pigment, preservative, protein, retinoid, reducing agent, sequesterant, skin bleaching agent, skin conditioning agent, skin smoothing agent, skin soothing agent, skin healing agent, softener, solubilizing agent, surfactant, lubricant, thickener, penetrant, permeation enhancer, analgesic, anti-inflammatory agent, antibiotic, anesthetic, plasticizer, salt, solvent essential oil, sunscreen and UV-absorber, vitamin, provitamine, plant extract, ceramide and pseudoceramide.

12. The personal care product as in any of claims 1 and 2, wherein said at least one personal care ingredient is present in an amount of no more than about 99.995% by weight of said product.

13. The personal care product of claim 12, wherein said at least one personal care ingredient is present in an amount of no more than about 99.99% by weight of said product.

14. The personal care product of claim 13 wherein said at least one personal care ingredient is present in an amount of no more than about 99.95% by weight of said product.

15. The personal care product of claim 14, wherein said at least one personal care ingredient is present in an amount of no more than about 99.9% by weight of said product.

16. The personal care product of claim 12, wherein said at least one personal care ingredient is present in an amount of at least about 10% by weight of said product.

17. The personal care product of claim 16, wherein said at least one personal care ingredient is present in an amount of at least about 60% by weight of said product.

18. The personal care product of claim 17, wherein said at least one personal care ingredient is present in an amount of at least about 80% by weight of said product.

19. The personal care product of claim 18, wherein said at least one personal care ingredient is present in an amount of at least about 90% by weight of said product.

20. The personal care product as in any one of claims 1 and 2, wherein said at least one EPOSS has at least two completed sides.

21. The personal care product of claim 20, wherein said EPOSS has no more than two Si groups bound to more than two R groups.

22. The personal care product of claim 2, formulated in the form of a liquid, solid, cream, ointment, solution, gel, mousse, stick, cream, spray, powder, emulsion or dispersion and wherein said at least one POSS is present in an amount of between about 0.01% and about 40% by weight of said product.

23. The personal care product of claims 1 or 2, wherein at least one R group is methyl, ethyl, propyl, isobutyl, isooctyl, phenyl, cyclohexyl, cyclopentyl, —OSi(CH₃)₂—CH₂—CH₂—(CF₂)₅CF₃, —(CH₂)₃SH, N⁺(CH₃)₃, O⁻ N⁺(CH₃)₃, —OH, —(CH₂)ₙN⁺H₃X⁻ wherein n is 0-30 and X is a counter ion,

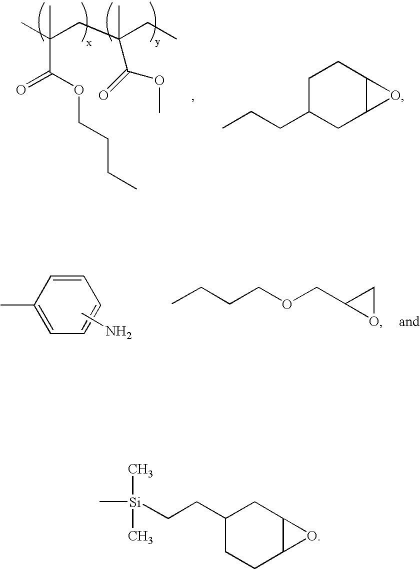

24. The personal care product of claim 23, wherein at least two of said R groups are methyl, ethyl, propyl, isobutyl, isooctyl, phenyl, cyclohexyl, cyclopentyl, —OSi(CH₃)₂—CH₂—CH₂—(CF₂)₅CF₃, —(CH₂)₃SH, N⁺(CH₃)₃, O⁻ N⁺(CH₃)₃, —OH, —(CH₂)ₙN⁺H₃X⁻ wherein n is 0-30 and X is a counter ion,

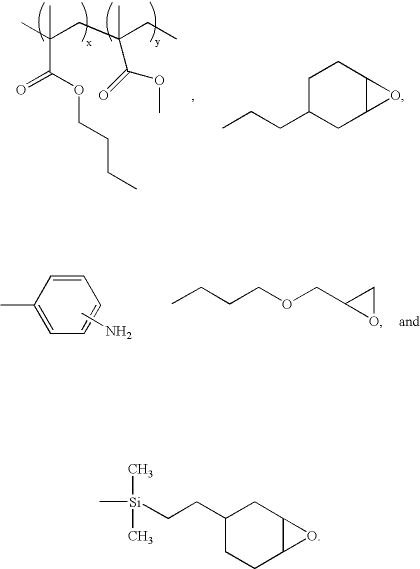

25. The personal care product of claim 24, wherein at least seven of said R groups are methyl, ethyl, propyl, isobutyl, isooctyl, phenyl, cyclohexyl, cyclopentyl, —OSi(CH₃)₂—CH₂—CH₂—(CF₂)₅CF₃, —(CH₂)₃SH, N⁺(CH₃)₃, O⁻ N⁺(CH₃)₃, —OH, —(CH₂)ₙN⁺H₃X⁻ wherein n is 0-30 and X is a counter ion,

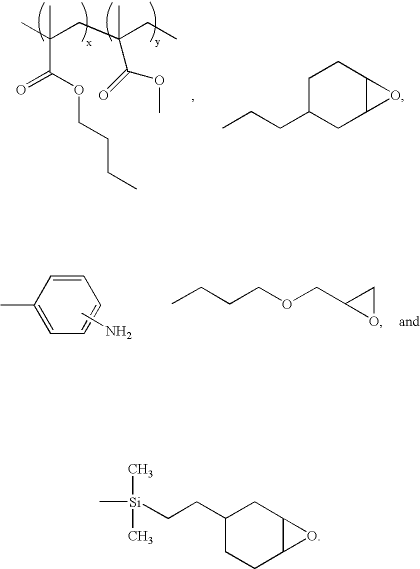

26. The personal care product as in any of claims 1 or 2, wherein said predetermined personal care property imparted by said EPOSS is conditioning, substantivity, solubility, comparability, film forming, coloring, permeation, adhesion, structure, and UV protection.

27. A shampoo comprising: at least one EPOSS having a rigid three dimensional cage structure comprising nine or more Si atoms within its cage structure, said EPOSS being present in an amount effective to provide a desired degree of a predetermined personal care property; and at least one personal care ingredient.

28. A nail polish comprising: at least one EPOSS having a rigid three dimensional cage structure comprising nine or more Si atoms within its cage structure, said EPOSS being present in an amount effective to provide a desired degree of a predetermined personal care property; and at least one personal care ingredient.

29. A conditioner comprising: at least one EPOSS having a rigid three dimensional cage structure comprising nine or more Si atoms within its cage structure, said EPOSS being present in an amount effective to provide a desired degree of a predetermined personal care property; and at least one personal care ingredient.

30. A mascara comprising: at least one EPOSS having a rigid three dimensional cage structure comprising nine or more Si atoms within its cage structure, said EPOSS being present in an amount effective to provide a desired degree of a predetermined personal care property; and at least one personal care ingredient.
